(19) United States
(12) Patent Application Publication
(10) Pub. No.: US 2025/0243427 A1
(43) Pub. Date: Jul. 31, 2025

(54) PRECURSORS OF PHENOLIC FRAGRANT COMPOUNDS

(71) Applicant: GIVAUDAN SA, Vernier (CH)

(72) Inventors: Lijun ZHOU, Shanghai (CN); Nicolas COCITO ARMANlNO, Kemptthal (CH); An CHAI, Shanghai (CN); Felix FLACHSMANN, Kemptthal (CH); Marc LINIGER, Kemptthal (CH)

(21) Appl. No.: 18/703,357

(22) PCT Filed: Oct. 20, 2022
(86) PCT No.: PCT/EP2022/079165

§ 371 Date: Apr. 21, 2024

(30) Foreign Application Priority Data
Oct. 21, 2021 (WO) PCT/CN2021/125195

(51) Int. Cl. C11B 9/00; A61K 8/37; A61Q 5/02; C07C 37/56

(52) U.S. Cl. CPC C11B 9/0061; C07C 37/56; A61K 8/375; A61Q 5/02

(57) ABSTRACT

There is provided a compound of formula (I)

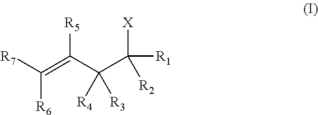

which is a homoallylic ether of a phenolic fragrant compound HX and which is able to release said phenolic fragrant compound HX.

### TECHNICAL FIELD

The present invention relates generally to fragrance precursors, which are able to release phenolic fragrant compounds. The invention also relates to perfume preparations and consumer products containing said precursors. The invention further relates to methods of making said perfume precursors, perfume preparations and consumer products, as well as the use of said perfume precursors and perfume preparations in consumer products, such as personal care and household care products.

### BACKGROUND

Perfumed consumer products such as cleaning or laundry products comprising fragrances are well-known in the art. However, it is known that fragrances can be altered when incorporated in certain consumer product bases, where alkalinity, acidity, the presence of oxidizing agents, such as hypochlorite salts, or other base components may lead to chemical degradation of the fragrance. In addition, volatile fragrances tend to be dissipated with time. Furthermore, when used in cleaning or laundry products, the deposition of the fragrance on a treated substrate is diminished by the washing and/or rinsing procedure.

In addition, in case of phenolic fragrant compounds like vanillin or ethyl vanillin, some discoloration issues in media like detergent bases can occur. Furthermore, phenolic fragrant compounds show a low substantivity in rinse-off applications due to their relatively high water solubility.

Nevertheless, it is desired by consumers to have products that can be stored overtime and still giving a constant perfume impression without discoloration. In particular, the impact of volatile components is to be retained. Furthermore, it is desired that such products create a long-lasting pleasing fragrance slowly emitting from the treated substrate overtime.

To address these needs, fragrance precursors can be used, which are substances that are typically odorless themselves, but which, in particular circumstances, will decompose to release the fragrant molecule.

There are several classes of known precursors which release fragrant molecules upon activation, such as hydrolysis, temperature change, oxygen, action of light and enzymes. For example, WO2012085287 reports a group of precursors able to release a fragrance by spontaneous air oxidation. In WO2007143873 another group of precursors is described which can be cleaved by hydrolysis.

All precursors do show different stability, and they release the fragrant molecule under different conditions. It is therefore desirable to provide a new or improved system which is capable of releasing phenolic fragrant compounds with a significant substantivity while avoiding discoloration.

### SUMMARY

In accordance with a first aspect of the present invention there is provided a use of a compound as fragrance precursor which is capable of releasing phenolic fragrant compounds.

In accordance with a second aspect of the present invention there are provided compounds as fragrance precursors.

In accordance with a third aspect of the present invention there are provided fragrance compositions and consumer products comprising said fragrance precursors.

In accordance with a fourth aspect of the present invention there is provided a method to release a phenolic fragrant compound.

In accordance with a fifth aspect of the present invention there is provided a method of making said fragrance precursors, and fragrance compositions and consumer products comprising said fragrance precursors.

In accordance with a sixth aspect of the present invention there is provided a method to confer, enhance, improve or modify the hedonic properties of a fragrance composition or a consumer product by using said fragrance precursors.

Certain embodiments of any aspect of the present invention may provide one or more of the following advantages:
release of phenolic fragrant compounds,
efficient delivery of the odors of phenolic fragrant compounds in consumer products,
stability of fragrance precursors in consumer product bases as well as after their deposition on substrates,
avoidance or great reduction of discoloration effects in comparison to accords using the free phenols.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based on the surprising finding that homoallyl ethers of phenolic fragrant compounds can serve as fragrance precursors able to release said phenolic fragrant compounds. These fragrance precursors provide a higher substantivity of the phenolic fragrant compounds while avoiding discoloration issues typically occurring when using free phenolic fragrant compounds.

There is therefore provided herein a use of a compound of formula (I)

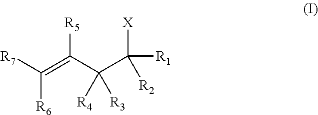

wherein
R₁-R₇ is independently selected from the group consisting of H or linear or branched C1-C4 hydrocarbon groups, bearing up to 2 heteroatoms selected from the group consisting of O, S and N, and bearing up to one double bond or triple bond, and bearing up to one ring; or
R₁ and R₆, R₁ and R₃, R₁ and R₅, R₃ and R₅, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, while the other substituents have the same meaning as previously defined;
and the sum of all carbon atoms of R₁-R₇ is limited to 4;
or R₇ is —CR′₃R′₄—CR′₁R′₂—X′, wherein R′₁, R′₂, R′₃, R′₄, and X can be selected from the corresponding groups of R₁, R₂, R₃, R₄, and X, while the other substituents have the same meaning as previously defined, and the sum of all carbon atoms of R₁-R₆ and R′₁-R′₄ is limited to 4;
and wherein X is representing the following fragment

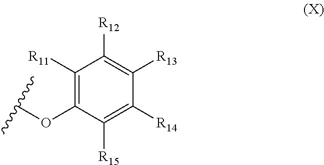

wherein
R₁₁-R₁₅ is independently selected from the group consisting of H, linear or branched C1-C5 alkyl, linear or branched C2-C5 alkenyl, linear or branched C2-C5 alkynyl, OH, C1-C4-alkoxy, CHO, C(O)Me, C(O)Et, hexanoyl, heptanoyl, octanoyl, propanoyl, 3-phenylpropanoyl, 5-methylhexanoyl, (CH₂)₂C(O)CH₃, (4-methyl-3,6-dihydro-2H-pyran-2-yl), CH₂—O-Me, CH₂—O-Et, CH₂—O-iPr, CH₂—OH, Cl, Br, phenyl, CH(OR₁₆)(OR₁₇) wherein R₁₆ and R₁₇ are independently selected from the group consisting of C1-C5 alkyl or R₁₆ and R₁₇ form together with the carbon atoms of the chain they are attached to a 5 or 6 membered ring, C═N—OR₁₈ wherein R₁₈ is C1-C5 alkyl, CO₂R₁₉ wherein R₁₉ is selected from the group consisting of linear or branched C1-C8 alkyl, linear or branched C2-C8 alkenyl, linear or branched C2-C8 alkynyl, cycloalkyl and aryl;
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms;
as precursor for generating a phenolic fragrant compound HX which is a compound of formula (II)

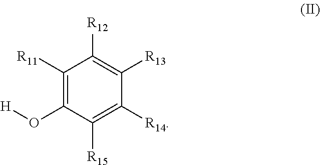

For example, there is provided a use of a compound of formula (I)

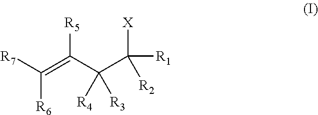

wherein
R₁-R₂ is independently selected from the group consisting of H, Me, Et, vinyl, ethynyl, allyl, iso-Pr, n-Pr, propenyl, propynyl, 2-methylprop-1-enyl, methylcarboxylate, ethylcarboxylate, acetyl,
R₃-R₅ is independently selected from the group consisting of H, Me, Et, iso-Pr, n-Pr, iso-Bu, n-Bu, tert-Bu, sec-Bu; and
R₆-R₇ is independently selected from the group consisting of H, Me, Et; iso-Pr, n-Pr, iso-Bu, n-Bu, tert-Bu, sec-Bu, propenyl, propynyl, methoxymethyl, cyclopropyl, cyclopropylmethyl, cyclobutyl, methylcarboxylate, ethylcarboxylate, carbaldehyde, hydroxymethyl, 1,3-butadienyl, 2-methylallyl, vinyl, ethynyl, acetyl; or
R₁ and R₆, R₁ and R₃, R₁ and R₅, R₃ and R₅, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, while the other substituents have the same meaning as previously defined;
and the sum of all carbon atoms of R₁-R₇ is limited to 4;
or R₇ is —CR′₃R′₄—CR′₁R′₂—X′, wherein R′₁, R′₂, R′₃, R′₄, and X can be selected from the corresponding groups of R₁, R₂, R₃, R₄, and X, and the sum of all carbon atoms of R₁-R₆ and R′₁-R′₄ is limited to 4;
and wherein X is representing the following fragment

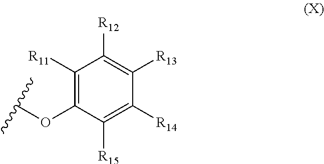

wherein
R₁₁ is selected from the group consisting of H, C1-C5 alkyl, OH, OMe, OEt, O-n-Pr, O-iso-Pr, hexanoyl, heptanoyl, octanoyl, propanoyl, 3-phenylpropanoyl, 5-methylhexanoyl, Cl, Br, phenyl, CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, para-tolyl;
R₁₂ is selected from the group consisting of H, OH, C1-C5 alkyl, C2-C5 alkenyl, C1-C5 alkoxy;
R₁₃ is selected from the group consisting of H, branched or linear C1-C5 alkyl, branched or linear C2-C5 alkenyl, CHO, CO₂H, CO₂Me, CO₂Et, C(O)Me, C(O)Et, (CH₂)₂C(O)CH₃; (4-methyl-3,6-dihydro-2H-pyran-2-yl), CH₂—O-Me, CH₂—O-Et, CH₂—O-iPr, CH₂—OH, Cl, propanoyl, CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, para-tolyl
R₁₄ is selected from the group consisting of H, OH, C1-C5 alkyl, C2-C5 alkenyl, C1-C5 alkoxy;
R₁₅ is selected from the group consisting of H, C1-C5 alkyl, Br,
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms,
as precursor for generating a phenolic fragrant compound HX which is a compound of formula (II)

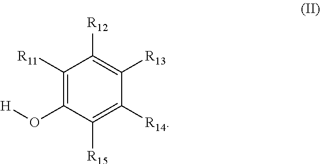

If not further specified, the CC double bond in the compound of formula (I) has either E- or Z-configuration, or the compound is a mixture of E and Z isomers.

The compounds of formula (I) can have one or more stereo centers and in consequence exist as different stereoisomers (e.g. diastereomers or enantiomers), all of which are encompassed by the present invention. Chiral compounds of formula (I) can be enantiomerically pure, enriched or racemic. If more than one stereo centre is present, the compounds are either diastereomeric mixtures, diastereomerically enriched or pure diastereomers.

The precursor effect of homoallyl ethers (compounds of formula (I)) is surprising, since allyl ethers and ethers with saturated side chains deliver significantly inferior performance.

The compound of formula (I) typically has low odour, when freshly prepared, and is able to release phenolic fragrant compound HX of formula (II), which is the dominant odor compound generated by the homoallyl ether precursor. Typically, the remaining part of the compound of formula (I) is either not contributing to the overall odor at all, or its contribution is relatively small, so that the main odor characteristics of the phenolic fragrant compound HX of formula (II) is perceived, and only side aspects of the odor are altered.

However, in some embodiments of the present invention, the compound of formula (I) might have an own odor.

By the use of the compound of formula (I) instead of the phenolic fragrant compound HX as such, it is possible to provide the odor of the phenolic fragrant compound HX while avoiding discoloration issues in a consumer product and/or on the surface, on which the consumer product is applied.

In addition, by the use of the compound of formula (I) instead of the phenolic fragrant compound HX as such, it is possible to provide the odor of the phenolic fragrant compound HX with an enhanced intensity, as the precursor remain on a surface, where it was deposited until the release of the phenolic fragrant compound HX. In contrast, the phenolic fragrant compound HX is for example washed away in rinse off applications due to the high water solubility of the compound.

Some homoallyl ethers are reported in literature. For example, JPS5412309A reports cis-hexenyl ethers of particular cresols as fragrance compounds. However, their use as fragrance precursors able to generate a phenolic fragrant compound HX is not disclosed.

For example, C1-C5 alkyl can be selected from the group consisting of methyl, ethyl, n-propyl, iso-propyl, n-butyl, iso-butyl, sec-butyl, tert-butyl, n-pentyl, iso-pentyl, tert-pentyl, neo-pentyl, 1-methylbutyl, 1-ethylpropyl, 1,2-dimethylpropyl and 2-methylbutyl.

For example, C2-C5 alkenyl can be selected from vinyl, C3 alkenyl like allyl, prop-1-en-2-yl, prop-1-en-1-yl, prop-2-en-2-yl, C4 alkenyl like but-1-en-1-yl, but-2-en-1-yl, but-3-en-1-yl, but-2-en-2-yl, 2-methylprop-1-en-1-yl, 2-methylallyl, but-3-en-2-yl, but-1-en-2-yl and others, C5 alkenyl like pent-1-en-1-yl, pent-2-en-1-yl, pent-3-en-1-yl, pent-4-en-1-yl, pent-2-en-2-yl, pent-3-en-2-yl, pent-4-en-2-yl, 3-methylbut-1-en-2-yl, and others.

According to one aspect of the invention, there is provided the use of the compound of formula (I) as precursor for generating a phenolic fragrant compound HX, with R₁-R₇ as defined above, and wherein fragment X is not o-, m-, or p-cresol, in which one of the substituents R₁₁-R₁₅ is Me, while the other substituents are H.

According to another aspect of the invention, there is provided the use of the compound of formula (I) as precursor for generating a phenolic fragrant compound HX, with R₁-R₇ as defined above, and wherein fragment X is not mono alkylated, in that one of the substituents R₁₁-R₁₅ is C1-C5 alkyl, while the other substituents are H.

For example, there is provided the use of the compound of formula (I) as precursor for generating a phenolic fragrant compound HX, wherein the residue attached to the fragment X is selected from the group consisting of cis-3 hexenyl, trans-3-hexenyl, cis- or trans-pentenyl, prenyl (3-methyl-2-butenyl) and cyclopent-3-enyl.

There is further provided the use of the compound of formula (I) as precursor for generating a phenolic fragrant compound HX, wherein the residue attached to the fragment X is selected from the group consisting of 4-heptenyl, 3-butenyl, 3-methyl-3-butenyl, 3-octenyl, 4-methyl-3-pentenyl, 1-heptenyl, 1,6-heptadienyl, 6-methyl-1,5-heptadienyl, 5-hexenyl, 1,5-heptadienyl, 2-methyl-4-pentenyl, 6-methyl-3-heptenyl, 5-methyl-3-hexenyl), 3-pentenyl, 4-methyl-3-hexenyl, 4-methyl-3-heptenyl, 4-methyl-3,6-heptadienyl, 4-methyl-3,5-heptadienyl. If the double bond in the residue is not in a terminal position or symmetrically substituted, the double bond can have either cis- or trans configuration, or the residue is a mixture of cis- and trans-isomers

According to another aspect of the invention, there is provided the use of the compound of formula (I) as precursor for generating a phenolic fragrant compound HX of formula (II) that can be released from the compound of formula (I), wherein the fragment X is derived from a phenolic fragrant compound HX selected from the group consisting of Vanillin (4-hydroxy-3-methoxybenzaldehyde), Ethyl vanillin (3-ethoxy-4-hydroxybenzaldehyde), Eugenol (4-allyl-2-methoxyphenol), Isoeugenol ((E)-2-methoxy-4-(prop-1-en-1-yl)phenol), Raspberry ketone (4-(4-hydroxyphenyl)butan-2-one), Evernyl (methyl 2,4-dihydroxy-3,6-dimethylbenzoate), Seamoss (methyl 2,4-dihydroxy-3-methylbenzoate), Cresol (2-methylphenol, 3-methylphenol, or 4-methylphenol), 4-ethyl phenol, 3-propyl phenol, Ultravanil (2-ethoxy-4-methylphenol), Guaiacol (2-Methoxyphenol), Vinyl Guaiacol (4-Ethenyl-2-methoxyphenol), Methyl Diantilis (2-Ethoxy-4-(methoxymethyl)phenol), Thymol (5-Methyl-2-(propan-2-yl)phenol), Eugewhite (2-methoxy-4-(4-methyl-3,6-dihydro-2H-pyran-2-yl)phenol), Zingerone (4-(4-hydroxy-3-methoxyphenyl)butan-2-one), Karmaflor ((4Z)-hept-4-en-2-yl 2-hydroxybenzoate), Saffiano (3-methylbenzofuran-5-ol), Carvacrol (5-isopropyl-2-methylphenol), Creosol (2-methoxy-4-methylphenol), Orcinyl 3 (3-methoxy-5-methylphenol), Hexyl salicylate (hexyl 2-hydroxybenzoate), Cyclohexyl salicylate (cyclohexyl 2-hydroxybenzoate), Phenyl ethyl salicylate (phenethyl 2-hydroxybenzoate), Octyl salicylate (2-ethylhexyl 2-hydroxybenzoate), Yasminate (3-methylhex-2-en-1-yl 2-hydroxybenzoate), Ethyl salicylate (ethyl 2-hydroxybenzoate), Hexenyl-3-cis salicylate ((Z)-hex-3-en-1-yl 2-hydroxybenzoate), Isobutyl salicylate (isobutyl 2-hydroxybenzoate), Methyl salicylate (methyl 2-hydroxybenzoate), Vanitrop (2-ethoxy-5-(prop-1-en-1-yl)phenol), Methylpentyl salicylate (hexan-2-yl 2-hydroxybenzoate), Para-cresyl salicylate (p-tolyl 2-hydroxybenzoate), Prenyl salicylate (3-methylbut-2-en-1-yl 2-hydroxybenzoate), Amyl salicylate (pentyl 2-hydroxybenzoate), Benzyl salicylate (benzyl 2-hydroxybenzoate), Sakura salicylate (2-isopropoxyethyl 2-hydroxybenzoate), 2-ethoxyphenol, 1-(2-hydroxyphenyl)-3-phenylpropan-1-one, 1-(2-hydroxyphenyl)hexan-1-one and 1-(2-hydroxyphenyl)heptan-1-one.

According to one aspect of the invention, there is provided the use of the compound of formula (I) as precursor for generating a phenolic fragrant compound HX of formula (II) that can be released from the compound of formula (I), wherein the fragment X is derived from a phenolic fragrant compound HX selected from the group consisting of Vanillin (4-hydroxy-3-methoxybenzaldehyde), Ethyl vanillin (3-ethoxy-4-hydroxybenzaldehyde), Eugenol (4-allyl-2-methoxyphenol), Isoeugenol ((E)-2-methoxy-4-(prop-1-en-1-yl)phenol), Raspberry ketone (4-(4-hydroxyphenyl)butan-2-one), Evernyl (methyl 2,4-dihydroxy-3,6-dimethylbenzoate), Seamoss (methyl 2,4-dihydroxy-3-methylbenzoate), Cresol (2-methylphenol, 3-methylphenol, or 4-methylphenol), 4-ethyl phenol, 3-propyl phenol, Ultravanil (2-ethoxy-4-methylphenol), Guaiacol (2-Methoxyphenol), Vinyl Guaiacol (4-Ethenyl-2-methoxyphenol), Methyl Diantilis (2-Ethoxy-4-(methoxymethyl)phenol), Thymol (5-Methyl-2-(propan-2-yl)phenol), Eugewhite (2-methoxy-4-(4-methyl-3,6-dihydro-2H-pyran-2-yl)phenol), Zingerone (4-(4-hydroxy-3-methoxyphenyl)butan-2-one), Karmaflor ((4Z)-hept-4-en-2-yl 2-hydroxybenzoate), Saffiano (3-methylbenzofuran-5-ol), Carvacrol (5-isopropyl-2-methylphenol), Creosol (2-methoxy-4-methylphenol), Orcinyl 3 (3-methoxy-5-methylphenol), Hexyl salicylate (hexyl 2-hydroxybenzoate), Cyclohexyl salicylate (cyclohexyl 2-hydroxybenzoate), Phenyl ethyl salicylate (phenethyl 2-hydroxybenzoate), Octyl salicylate (2-ethylhexyl 2-hydroxybenzoate), Yasminate (3-methylhex-2-en-1-yl 2-hydroxybenzoate), Ethyl salicylate (ethyl 2-hydroxybenzoate), Hexenyl-3-cis salicylate ((Z)-hex-3-en-1-yl 2-hydroxybenzoate), Isobutyl salicylate (isobutyl 2-hydroxybenzoate), Methyl salicylate (methyl 2-hydroxybenzoate), Vanitrop (2-ethoxy-5-(prop-1-en-1-yl)phenol), Methylpentyl salicylate (hexan-2-yl 2-hydroxybenzoate), Para-cresyl salicylate (p-tolyl 2-hydroxybenzoate), Prenyl salicylate (3-methylbut-2-en-1-yl 2-hydroxybenzoate), Amyl salicylate (pentyl 2-hydroxybenzoate), Benzyl salicylate (benzyl 2-hydroxybenzoate), Sakura salicylate (2-isopropoxyethyl 2-hydroxybenzoate), 2-ethoxyphenol, 1-(2-hydroxyphenyl)-3-phenylpropan-1-one, 1-(2-hydroxyphenyl)hexan-1-one and 1-(2-hydroxyphenyl)heptan-1-one, and wherein the residue attached to the fragment X is selected from the group consisting of cis-3 hexenyl, trans-3-hexenyl, cis or trans-pentenyl, prenyl (4-methyl-3-butyl) and cyclopent-3-enyl.

For example, there is provided the use of the compound of formula (I) as precursor selected from the group consisting of (Z)-3-ethoxy-4-(hex-3-en-1-yloxy)benzaldehyde, (E)-3-ethoxy-4-(hex-3-en-1-yloxy)benzaldehyde, 3-ethoxy-4-(hex-3-en-1-yloxy)benzaldehyde, (Z)-3-ethoxy-4-(hept-4-en-2-yloxy)benzaldehyde, (Z)-4-(4-(hept-4-en-2-yloxy)phenyl)butan-2-one, (Z)-4-(4-(hex-3-en-1-yloxy)phenyl)butan-2-one, (E)-4-(4-(hex-3-en-1-yloxy)phenyl)butan-2-one, (Z)-4-(hex-3-en-1-yloxy)-3-methoxybenzaldehyde, methyl (Z)-4-(hex-3-en-1-yloxy)-2-hydroxy-3,6-dimethylbenzoate, (Z)-4-allyl-1-(hex-3-en-1-yloxy)-2-methoxybenzene, (Z)-4-allyl-1-(hex-3-en-1-yloxy)-2-methoxybenzene, 4-(but-3-en-1-yloxy)-3-ethoxybenzaldehyde, 4-allyl-1-(but-3-en-1-yloxy)-2-methoxybenzene, methyl 4-(but-3-en-1-yloxy)-2-hydroxy-3,6-dimethylbenzoate, 3-ethoxy-4-((3-methylbut-3-en-1-yl)oxy)benzaldehyde, 4-(4-((3-methylbut-3-en-1-yl)oxy)phenyl)butan-2-one, (Z)-3-ethoxy-4-(oct-3-en-1-yloxy)benzaldehyde, 4-(4-((4-methylpent-3-en-1-yl)oxy)phenyl)butan-2-one, 3-ethoxy-4-(hept-1-en-4-yloxy)benzaldehyde, 4-(cyclopent-3-en-1-yloxy)-3-ethoxybenzaldehyde, 4-(4-(hept-1-en-4-yloxy)phenyl)butan-2-one, 3-ethoxy-4-(hepta-1,6-dien-4-yloxy)benzaldehyde, 4-(4-(hepta-1,6-dien-4-yloxy)phenyl)butan-2-one, 3-ethoxy-4-((6-methylhepta-1,5-dien-4-yl)oxy)benzaldehyde, 4-(4-(hex-5-en-3-yloxy)phenyl)butan-2-one, 4-(4-(hepta-1,5-dien-4-yloxy)phenyl)butan-2-one, 4-(4-((6-methylhepta-1,5-dien-4-yl)oxy)phenyl)butan-2-one, 3-ethoxy-4-((2-methylpent-4-en-2-yl)oxy)benzaldehyde, 2-(hex-3-en-1-yloxy)-1-isopropyl-4-methylbenzene, 3-ethoxy-4-((6-methylhept-3-en-1-yl)oxy)benzaldehyde, (E)-3-ethoxy-4-((5-methylhex-3-en-1-yl)oxy)benzaldehyde, 3-ethoxy-4-(pent-3-en-1-yloxy)benzaldehyde, 3-ethoxy-4-((4-methylpent-3-en-1-yl)oxy)benzaldehyde, (Z)-2-ethoxy-1-(hex-3-en-1-yloxy)-4-methylbenzene, (Z)-1-ethoxy-2-(hex-3-en-1-yloxy)-4-methylbenzene, 4-(4-((4-methylhex-3-en-1-yl)oxy)phenyl)butan-2-one, 3-methoxy-4-((4-methylpent-3-en-1-yl)oxy)benzaldehyde, 3-methoxy-4-((4-methylhept-3-en-1-yl)oxy)benzaldehyde, (Z)-1-ethoxy-2-(hex-3-en-1-yloxy)benzene, 3-ethoxy-4-((4-methylhex-3-en-1-yl)oxy)benzaldehyde, (Z)-1-(hex-3-en-1-yloxy)-3-methoxy-5-methylbenzene, 1-ethyl-4-((4-methylpent-3-en-1-yl)oxy)benzene, 1-methoxy-3-methyl-5-((4-methylpent-3-en-1-yl)oxy)benzene, 1-methyl-4-((4-methylpent-3-en-1-yl)oxy)benzene, 1-((4-methylpent-3-en-1-yl)oxy)-3-propylbenzene, 3-ethoxy-4-((4-methylhepta-3,6-dien-1-yl)oxy)benzaldehyde, 3-ethoxy-4-((4-methylhepta-3,5-dien-1-yl)oxy)benzaldehyde, methyl 5-(2-ethoxy-4-formylphenoxy)pent-2-enoate, ethyl 2-(2-ethoxy-4-formylphenoxy)pent-4-enoate, methyl 2-hydroxy-3,6-dimethyl-4-((4-methylpent-3-en-1-yl)oxy)benzoate, methyl 2-hydroxy-3,6-dimethyl-4-(pent-3-en-1-yloxy)benzoate.

Furthermore, there is provided the use of the compound of formula (I) as precursor selected from the group consisting of methyl (Z)-4-(hex-3-en-1-yloxy)-2-hydroxy-3,6-dimethylbenzoate, methyl (E)-4-(hex-3-en-1-yloxy)-2-hydroxy-3,6-dimethylbenzoate, (E)-1-(cyclopent-3-en-1-yloxy)-2-methoxy-4-(prop-1-en-1-yl)benzene, methyl 4-(cyclopent-3-en-1-yloxy)-2-hydroxy-3,6-dimethylbenzoate, methyl 4-(hept-1-en-4-yloxy)-2-hydroxy-3,6-dimethylbenzoate, (E)-4-((2,2-dimethylpent-3-en-1-yl)oxy)-3-ethoxybenzaldehyde, methyl 4-(hepta-1,6-dien-4-yloxy)-2-hydroxy-3,6-dimethylbenzoate, (E)-3-ethoxy-4-(hepta-1,5-dien-4-yloxy)benzaldehyde, 3-ethoxy-4-(hex-5-en-3-yloxy)benzaldehyde, methyl 4-(hex-5-en-3-yloxy)-2-hydroxy-3,6-dimethylbenzoate, methyl 2-hydroxy-3,6-dimethyl-4-((6-methylhepta-1,5-dien-4-yl)oxy)benzoate, methyl (E)-4-(hepta-1,5-dien-4-yloxy)-2-hydroxy-3,6-dimethylbenzoate, 1-(cyclohex-3-en-1-yloxy)-2-ethoxy-4-methylbenzene, 4-(cyclohex-3-en-1-yloxy)-3-ethoxybenzaldehyde, (E)-4-(4-((4-methylhept-3-en-1-yl)oxy)phenyl)butan-2-one, (E)-3-ethoxy-4-((4-methylhept-3-en-1-yl)oxy)benzaldehyde, (E)-4-(4-(pent-3-en-1-yloxy)phenyl)butan-2-one, (E)-3-methoxy-4-(pent-3-en-1-yloxy)benzaldehyde, (E)-4-(hex-3-en-1-yloxy)-3-methoxybenzaldehyde.

Furthermore, there is provided the use of the compound of formula (I) as precursor, wherein the precursor is represented by the compound of formula (III)

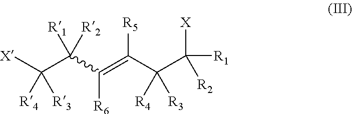

wherein
R₁-R₆ is independently selected from the group consisting of H or linear or branched C1-C4 hydrocarbon groups, bearing up to 2 heteroatoms selected from the group consisting of O, S and N, and bearing up to one double bond or triple bond, and bearing up to one ring; and
X is representing the fragment as defined above for the compound of formula (I), the wavy bond is indicating an unspecified configuration of the adjacent double bond,
and wherein R′₁, R′₂, R′₃, R′₄, and X can be selected from the corresponding groups of R₁, R₂, R₃, R₄, and X, respectively,
and the sum of all carbon atoms of R₁-R₆ and R′₁-R′₄ is limited to 4.

For example, there is provided the use of the compound of formula (I) as precursor, wherein the precursor is represented by the compound of formula (III)

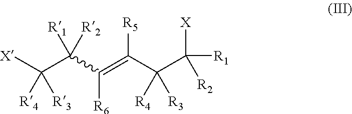

wherein
R₁-R₂ is independently selected from the group consisting of H, Me, Et, vinyl, ethynyl, allyl, iso-Pr, n-Pr, propenyl, propynyl, 2-methylprop-1-enyl, methylcarboxylate, ethylcarboxylate, acetyl,
R₃-R₅ is independently selected from the group consisting of H, Me, Et, iso-Pr, n-Pr, iso-Bu, n-Bu, tert-Bu, sec-Bu; and
R₆ is selected from the group consisting of H, Me, Et; iso-Pr, n-Pr, iso-Bu, n-Bu, tert-Bu, sec-Bu, propenyl, propynyl, methoxymethyl, cyclopropyl, cyclopropylmethyl, cyclobutyl, methylcarboxylate, ethylcarboxylate, carbaldehyde, hydroxymethyl, 1,3-butadienyl, 2-methylallyl, vinyl, ethynyl, acetyl;
X is representing the fragment as defined above for the compound of formula (I), the wavy bond is indicating an unspecified configuration of the adjacent double bond,
and wherein R′₁, R′₂, R′₃, R′₄, and X′ can be selected from the corresponding groups of R₁, R₂, R₃, R₄, and X, respectively,
and the sum of all carbon atoms of R₁-R₆ and R′₁-R′₄ is limited to 4.

The compound of formula (III) is able to release two phenolic fragrant compounds HX and HX′, which could be same or different.

While a few cis-hexenyl ethers of particular cresols are reported in JPS5412309A, most of the compounds of formula (I) are novel.

So in a further embodiment, there is provided the compound of formula (I)

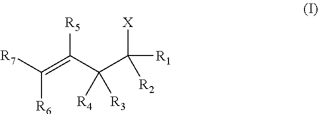

wherein
R₁-R₇ is independently selected from the group consisting of H or linear or branched C1-C4 hydrocarbon groups, bearing up to 2 heteroatoms selected from the group consisting of O, S and N, and bearing up to one double bond or triple bond, and bearing up to one ring; or
R₁ and R₆, R₁ and R₃, R₁ and R₅, R₃ and R₅, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, while the other substituents have the same meaning as previously defined;
and the sum of all carbon atoms of R₁-R₇ is limited to 4;
or R₇ is —CR′₃R′₄—CR′₁R′₂—X′, wherein R′₁, R′₂, R′₃, R′₄, and X′ can be selected from the corresponding groups of R₁, R₂, R₃, R₄, and X, while the other substituents have the same meaning as previously defined, and the sum of all carbon atoms of R₁-R₆ and R′₁-R′₄ is limited to 4;
and wherein X is representing the following fragment

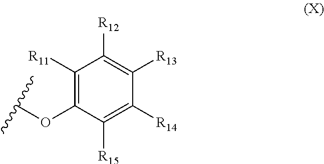

wherein
R₁₁-R₁₅ is independently selected from the group consisting of H, linear or branched C1-C5 alkyl, linear or branched C2-C5 alkenyl, linear or branched C2-C5 alkynyl, OH, C1-C4-alkoxy, CHO, C(O)Me, C(O)Et, hexanoyl, heptanoyl, octanoyl, propanoyl, 3-phenylpropanoyl, 5-methylhexanoyl, (CH₂)₂C(O)CH₃, (4-methyl-3,6-dihydro-2H-pyran-2-yl), CH₂—O-Me, CH₂—O-Et, CH₂—O-iPr, CH₂—OH, Cl, Br, phenyl, CH(OR₁₆)(OR₁₇) wherein R₁₆ and R₁₇ are independently selected from the group consisting of C1-C5 alkyl or R₁₆ and R₁₇ form together with the carbon atoms of the chain they are attached to a 5 or 6 membered ring, C═N—OR₁₈ (R₁₈═C1-C5 alkyl), CO₂R₁₉, wherein R₁₉ is selected from the group consisting of linear or branched C1-C8 alkyl, linear or branched C2-C8 alkenyl, linear or branched C2-C8 alkynyl, cycloalkyl and aryl,
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms;
with the proviso that the compound is not (Z)-1-(tert-butyl)-4-(hex-3-en-1-yloxy)benzene, (Z)-1-(hex-3-en-1-yloxy)-4-methylbenzene, (Z)-1-(hex-3-en-1-yloxy)-3-methylbenzene, (Z)-1-(hex-3-en-1-yloxy)-2-methylbenzene, or (Z)-(hex-3-en-1-yloxy)benzene.

For example, there is provided the compound of formula (I)

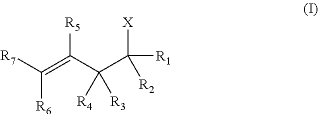

wherein
R₁-R₂ is independently selected from the group consisting of H, Me, Et, vinyl, ethynyl, allyl, iso-Pr, n-Pr, propenyl, propynyl, 2-methylprop-1-enyl, methylcarboxylate, ethylcarboxylate, acetyl,
R₃-R₅ is independently selected from the group consisting of H, Me, Et, iso-Pr, n-Pr, iso-Bu, n-Bu, tert-Bu, sec-Bu; and
R₆-R₇ is independently selected from the group consisting of H, Me, Et; iso-Pr, n-Pr, iso-Bu, n-Bu, tert-Bu, sec-Bu, propenyl, propynyl, methoxymethyl, cyclopropyl, cyclopropylmethyl, cyclobutyl, methylcarboxylate, ethylcarboxylate, carbaldehyde, hydroxymethyl, 1,3-butadienyl, 2-methylallyl, vinyl, ethynyl, acetyl; or
R₁ and R₆, R₁ and R₃, R₁ and R₅, R₃ and R₅, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, while the other substituents have the same meaning as previously defined;
and the sum of all carbon atoms of R₁-R₇ is limited to 4;
or R₇ is —CR′₃R′₄—CR′₁R′₂—X′, wherein R′₁, R′₂, R′₃, R′₄, and X′ can be selected from the corresponding groups of R₁, R₂, R₃, R₄, and X, and the sum of all carbon atoms of R₁-R₆ and R′₁-R′₄ is limited to 4;
and wherein X is representing the following fragment

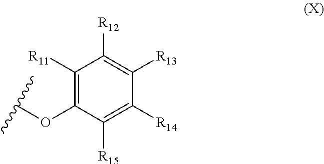

wherein
R₁₁ is selected from the group consisting of H, C1-C5 alkyl, OH, OMe, OEt, O-n-Pr, O-iso-Pr, hexanoyl, heptanoyl, octanoyl, propanoyl, 3-phenylpropanoyl, 5-methylhexanoyl, Cl, Br, phenyl, CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, para-tolyl;
R₁₂ is selected from the group consisting of H, OH, C1-C5 alkyl, C2-C5 alkenyl, C1-C5 alkoxy;
R₁₃ is selected from the group consisting of H, branched or linear C1-C5 alkyl, branched or linear C2-C5 alkenyl, CHO, CO₂H, CO₂Me, CO₂Et, C(O)Me, C(O)Et, (CH₂)₂C(O)CH₃; (4-methyl-3,6-dihydro-2H-pyran-2-yl), CH₂—O-Me, CH₂—O-Et, CH₂—O-iPr, CH₂—OH, Cl, propanoyl, CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, para-tolyl
R₁₄ is selected from the group consisting of H, OH, C1-C5 alkyl, C2-C5 alkenyl, C1-C5 alkoxy;
R₁₅ is selected from the group consisting of H, C1-C5 alkyl, Br,
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms,
with the proviso that the compound is not (Z)-1-(tert-butyl)-4-(hex-3-en-1-yloxy)benzene, (Z)-1-(hex-3-en-1-yloxy)-4-methylbenzene, (Z)-1-(hex-3-en-1-yloxy)-3-methylbenzene, (Z)-1-(hex-3-en-1-yloxy)-2-methylbenzene, or (Z)-(hex-3-en-1-yloxy)benzene.

For example, the compound of formula (I) can be selected from the group consisting of (Z)-3-ethoxy-4-(hex-3-en-1-yloxy)benzaldehyde, (E)-3-ethoxy-4-(hex-3-en-1-yloxy)benzaldehyde, 3-ethoxy-4-(hex-3-en-1-yloxy)benzaldehyde, (Z)-3-ethoxy-4-(hept-4-en-2-yloxy)benzaldehyde, (Z)-4-(4-(hept-4-en-2-yloxy)phenyl)butan-2-one, (Z)-4-(4-(hex-3-en-1-yloxy)phenyl)butan-2-one, (E)-4-(4-(hex-3-en-1-yloxy)phenyl)butan-2-one, (Z)-4-(hex-3-en-1-yloxy)-3-methoxybenzaldehyde, methyl (Z)-4-(hex-3-en-1-yloxy)-2-hydroxy-3,6-dimethylbenzoate, (Z)-4-allyl-1-(hex-3-en-1-yloxy)-2-methoxybenzene, (Z)-4-allyl-1-(hex-3-en-1-yloxy)-2-methoxybenzene, 4-(but-3-en-1-yloxy)-3-ethoxybenzaldehyde, 4-allyl-1-(but-3-en-1-yloxy)-2-methoxybenzene, methyl 4-(but-3-en-1-yloxy)-2-hydroxy-3,6-dimethylbenzoate, 3-ethoxy-4-((3-methylbut-3-en-1-yl)oxy)benzaldehyde, 4-(4-((3-methylbut-3-en-1-yl)oxy)phenyl)butan-2-one, (Z)-3-ethoxy-4-(oct-3-en-1-yloxy)benzaldehyde, 4-(4-((4-methylpent-3-en-1-yl)oxy)phenyl)butan-2-one, 3-ethoxy-4-(hept-1-en-4-yloxy)benzaldehyde, 4-(cyclopent-3-en-1-yloxy)-3-ethoxybenzaldehyde, 4-(4-(hept-1-en-4-yloxy)phenyl)butan-2-one, 3-ethoxy-4-(hepta-1,6-dien-4-yloxy)benzaldehyde, 4-(4-(hepta-1,6-dien-4-yloxy)phenyl)butan-2-one, 3-ethoxy-4-((6-methylhepta-1,5-dien-4-yl)oxy)benzaldehyde, 4-(4-(hex-5-en-3-yloxy)phenyl)butan-2-one, 4-(4-(hepta-1,5-dien-4-yloxy)phenyl)butan-2-one, 4-(4-((6-methylhepta-1,5-dien-4-yl)oxy)phenyl)butan-2-one, 3-ethoxy-4-((2-methylpent-4-en-2-yl)oxy)benzaldehyde, 2-(hex-3-en-1-yloxy)-1-isopropyl-4-methylbenzene, 3-ethoxy-4-((6-methylhept-3-en-1-yl)oxy)benzaldehyde, (E)-3-ethoxy-4-((5-methylhex-3-en-1-yl)oxy)benzaldehyde, 3-ethoxy-4-(pent-3-en-1-yloxy)benzaldehyde, 3-ethoxy-4-((4-methylpent-3-en-1-yl)oxy)benzaldehyde, (Z)-2-ethoxy-1-(hex-3-en-1-yloxy)-4-methylbenzene, (Z)-1-ethoxy-2-(hex-3-en-1-yloxy)-4-methylbenzene, 4-(4-((4-methylhex-3-en-1-yl)oxy)phenyl)butan-2-one, 3-methoxy-4-((4-methylpent-3-en-1-yl)oxy)benzaldehyde, 3-methoxy-4-((4-methylhept-3-en-1-yl)oxy)benzaldehyde, (Z)-1-ethoxy-2-(hex-3-en-1-yloxy)benzene, 3-ethoxy-4-((4-methylhex-3-en-1-yl)oxy)benzaldehyde, (Z)-1-(hex-3-en-1-yloxy)-3-methoxy-5-methylbenzene, 1-ethyl-4-((4-methylpent-3-en-1-yl)oxy)benzene, 1-methoxy-3-methyl-5-((4-methylpent-3-en-1-yl)oxy)benzene, 1-methyl-4-((4-methylpent-3-en-1-yl)oxy)benzene, 1-((4-methylpent-3-en-1-yl)oxy)-3-propylbenzene, 3-ethoxy-4-((4-methylhepta-3,6-dien-1-yl)oxy)benzaldehyde and 3-ethoxy-4-((4-methylhepta-3,5-dien-1-yl)oxy)benzaldehyde, methyl (Z)-4-(hex-3-en-1-yloxy)-2-hydroxy-3,6-dimethylbenzoate, methyl (E)-4-(hex-3-en-1-yloxy)-2-hydroxy-3,6-dimethylbenzoate, (E)-1-(cyclopent-3-en-1-yloxy)-2-methoxy-4-(prop-1-en-1-yl)benzene, methyl 4-(cyclopent-3-en-1-yloxy)-2-hydroxy-3,6-dimethylbenzoate, methyl 4-(hept-1-en-4-yloxy)-2-hydroxy-3,6-dimethylbenzoate, (E)-4-((2,2-dimethylpent-3-en-1-yl)oxy)-3-ethoxybenzaldehyde, methyl 4-(hepta-1,6-dien-4-yloxy)-2-hydroxy-3,6-dimethylbenzoate, (E)-3-ethoxy-4-(hepta-1,5-dien-4-yloxy)benzaldehyde, 3-ethoxy-4-(hex-5-en-3-yloxy)benzaldehyde, methyl 4-(hex-5-en-3-yloxy)-2-hydroxy-3,6-dimethylbenzoate, methyl 2-hydroxy-3,6-dimethyl-4-((6-methylhepta-1,5-dien-4-yl)oxy)benzoate, methyl (E)-4-(hepta-1,5-dien-4-yloxy)-2-hydroxy-3,6-dimethylbenzoate, 1-(cyclohex-3-en-1-yloxy)-2-ethoxy-4-methylbenzene, 4-(cyclohex-3-en-1-yloxy)-3-ethoxybenzaldehyde, (E)-4-(4-((4-methylhept-3-en-1-yl)oxy)phenyl)butan-2-one, (E)-3-ethoxy-4-((4-methylhept-3-en-1-yl)oxy)benzaldehyde, (E)-4-(4-(pent-3-en-1-yloxy)phenyl)butan-2-one, (E)-3-methoxy-4-(pent-3-en-1-yloxy)benzaldehyde, (E)-4-(hex-3-en-1-yloxy)-3-methoxybenzaldehyde, methyl 5-(2-ethoxy-4-formylphenoxy)pent-2-enoate, ethyl 2-(2-ethoxy-4-formylphenoxy)pent-4-enoate, methyl 2-hydroxy-3,6-dimethyl-4-((4-methylpent-3-en-1-yl)oxy)benzoate, methyl 2-hydroxy-3,6-dimethyl-4-(pent-3-en-1-yloxy)benzoate.

The compounds of formula (I) described above release phenolic fragrant compounds HX of formula (II) upon exposure to ambient air by oxidation in the presence or absence of light over a long period of time, e.g. several days such as 2-7 days or even longer.

In one embodiment, the compounds of the present invention can be perceived in the presence of malodour. Additionally, the compounds are able to reduce the perceived intensity of malodour. For example, the malodour can be sweat malodour, dank or musty smell, for example on drying fabrics, bathroom malodour, kitchen/food malodour or stale tobacco.

In a further aspect of the present invention, there is provided a method to release a phenolic fragrant compound of formula (II), wherein a compound of formula (I) is exposed to ambient air in the presence or absence of light.

For example, the phenolic fragrant compound HX of formula (II) that can be released from the compound of formula (I) upon exposure to ambient air in the presence or absence of light can be selected from the group consisting of Vanillin (4-hydroxy-3-methoxybenzaldehyde), Ethyl vanillin (3-ethoxy-4-hydroxybenzaldehyde), Eugenol (4-allyl-2-methoxyphenol), Isoeugenol ((E)-2-methoxy-4-(prop-1-en-1-yl)phenol), Raspberry ketone (4-(4-hydroxyphenyl)butan-2-one), Evernyl (methyl 2,4-dihydroxy-3,6-dimethylbenzoate), Seamoss (methyl 2,4-dihydroxy-3-methylbenzoate), Cresol (2-methylphenol, 3-methylphenol, or 4-methylphenol), 4-ethyl phenol, 3-propyl phenol, Ultravanil (2-ethoxy-4-methylphenol), Guaiacol (2-Methoxyphenol), Vinyl Guaiacol (4-Ethenyl-2-methoxyphenol), Methyl Diantilis (2-Ethoxy-4-(methoxymethyl)phenol), Thymol (5-Methyl-2-(propan-2-yl)phenol), Eugewhite (2-methoxy-4-(4-methyl-3,6-dihydro-2H-pyran-2-yl)phenol), Zingerone (4-(4-hydroxy-3-methoxyphenyl)butan-2-one), Karmaflor ((4Z)-hept-4-en-2-yl 2-hydroxybenzoate), Saffiano (3-methylbenzofuran-5-ol), Carvacrol (5-isopropyl-2-methylphenol), Creosol (2-methoxy-4-methylphenol), Orcinyl 3 (3-methoxy-5-methylphenol), Hexyl salicylate (hexyl 2-hydroxybenzoate), Cyclohexyl salicylate (cyclohexyl 2-hydroxybenzoate), Phenyl ethyl salicylate (phenethyl 2-hydroxybenzoate), Octyl salicylate (2-ethylhexyl 2-hydroxybenzoate), Yasminate (3-methylhex-2-en-1-yl 2-hydroxybenzoate), Ethyl salicylate (ethyl 2-hydroxybenzoate), Hexenyl-3-cis salicylate ((Z)-hex-3-en-1-yl 2-hydroxybenzoate), Isobutyl salicylate (isobutyl 2-hydroxybenzoate), Methyl salicylate (methyl 2-hydroxybenzoate), Vanitrop (2-ethoxy-5-(prop-1-en-1-yl)phenol), Methylpentyl salicylate (hexan-2-yl 2-hydroxybenzoate), Para-cresyl salicylate (p-tolyl 2-hydroxybenzoate), Prenyl salicylate (3-methylbut-2-en-1-yl 2-hydroxybenzoate), Amyl salicylate (pentyl 2-hydroxybenzoate), Benzyl salicylate (benzyl 2-hydroxybenzoate), Sakura salicylate (2-isopropoxyethyl 2-hydroxybenzoate), 2-ethoxyphenol, 1-(2-hydroxyphenyl)-3-phenylpropan-1-one, 1-(2-hydroxyphenyl)hexan-1-one, 1-(2-hydroxyphenyl)heptan-1-one.

For example, the phenolic fragrant compound HX of formula (II) that can be released from the compound of formula (I) upon on exposure to ambient air in the presence or absence of light is a phenol having at least one substituent at the aromatic ring, the at least one substituent having 1 carbon atom or more, for example 2 to 4 carbon atoms or more.

The compounds of formula (I) release phenolic fragrant compounds HX of formula (II) upon exposure to ambient air in the presence or absence of light over a long period of time (e.g. several days such as 2-7 days or even longer).

Exposure of the precursor compound to ambient air means exposure to molecular oxygen which might be responsible for the oxidative cleavage of the compound of formula (I) and the release of the compound of formula (II). The concentration of oxygen in the air is sufficient for cleaving the compound of formula (I) so that the cleavage products can be detected in the ambient air, e.g. by olfaction or GC-MS analysis of collected organic volatiles from the precursor headspace.

The compounds of formula (I) are very stable when their exposure to ambient air is limited or prevented, i.e. when stored in neat form in appropriate containers protected from air and light, or when stored in proper solvents, for example in ethanol, isopropanol, diethylene glycol monoethyl ether, glycerol, propylene glycol, 1,2-butylene glycol, dipropylene glycol, isopropyl myristate, triethyl citrate, diethyl phthalate, triacetine and/or diacetine, or when incorporated in consumer products such as detergent, shampoo and fabric conditioner. Thus the compounds of formula (I) may find use in a broad range of consumer products in which a prolonged and defined release of fragrant compounds is desired.

In some embodiments, stabilizing compounds, for example alpha tocopherol, EDTA, ascorbic acid, BHT, Tinoguard TT, can be added to the compounds of formula (I), for example in 0.01-1% by weight, to limit or prevent premature cleavage of the compound of formula (I) and the release of the compound of formula (II). In particular, the stabilizing compounds can be used to enhance the stability of the neat compounds of formula (I).

The compound of formula (I) may be used alone, or in combination with known odorant molecules selected from the extensive range of natural products, and synthetic molecules currently available, such as essential oils, alcohols, aldehydes and ketones, ethers and acetals, esters and lactones, macrocycles and heterocycles, and/or in admixture with one or more ingredients or excipients conventionally used in conjunction with odorants in perfume compositions, for example, carrier materials, and other auxiliary agents commonly used in the art.

For example, the compound of formula (I) may be used in combination with the free phenolic fragrant compound HX. Such a combination ensures a continuous perception of the phenolic fragrant compound HX with time. It is particularly useful, if the discoloration effect of free phenolic fragrant compound HX is not prominent or is not relevant for the consumer.

In a further aspect, the compound of formula (I) may be used in combination with other fragrance precursors, either with further compounds of formula (I) or with precursors possessing a different chemical structure. A combination of precursors allows releasing a perfume accord.

In a further aspect, there is provided a fragrance composition comprising at least one compound of formula (I). For example, the fragrance composition is further comprising one or more known odourant molecules, and/or one or more ingredients or excipients conventionally used in conjunction with odourants in perfume compositions

As used herein, “carrier material” means a material which is practically neutral from a odourant point of view, i.e. a material that does not significantly alter the organoleptic properties of odourants.

The term “auxiliary agent” refers to ingredients that might be employed in a fragrance composition for reasons not specifically related to the olfactive performance of said composition. For example, an auxiliary agent may be an ingredient that acts as an aid to processing a fragrance ingredient or ingredients, or a composition containing said ingredient(s), or it may improve handling or storage of a fragrance ingredient or composition containing same. It might also be an ingredient that provides additional benefits such as imparting color or texture. It might also be an ingredient that imparts light resistance or chemical stability to one or more ingredients contained in a perfume composition. A detailed description of the nature and type of adjuvants commonly used in perfume compositions containing same cannot be exhaustive, but it has to be mentioned that said ingredients are well known to a person skilled in the art.

As used herein, “fragrance composition” means any composition comprising the compound of formula (I) and a base material, e.g. a diluent conventionally used in conjunction with odourants, such as diethyl phthalate (DEP), dipropylene glycol (DPG), isopropyl myristate (IPM), pentane-1,2-diol, triethyl citrate (TEC) and alcohol (e.g. ethanol). Optionally, the composition may comprise an anti-oxidant adjuvant. Said anti-oxidant may be selected from Tinogard® TT (BASF), Tinogard® Q (BASF), Tocopherol (including its isomers, CAS 59-02-9; 364-49-8; 18920-62-2; 121854-78-2), 2,6-bis(1,1-dimethylethyl)-4-methylphenol (BHT, CAS 128-37-0) and related phenols, hydroquinones (CAS 121-31-9).

The following non limiting list comprises examples of known odourant molecules, which may be combined with the compound of formula (I) in a fragrance composition:
Essential oils and extracts, e.g. castoreum, costus root oil, oak moss absolute, geranium oil, tree moss absolute, basil oil, fruit oils, such as bergamot oil and mandarine oil, myrtle oil, palmarose oil, patchouli oil, petitgrain oil, jasmine oil, rose oil, sandalwood oil, wormwood oil, lavender oil and/or ylang-ylang oil;
Alcohols, e.g. cinnamic alcohol ((E)-3-phenylprop-2-en-1-ol); cis-3-hexenol ((Z)-hex-3-en-1-ol); Citronellol (3,7-dimethyloct-6-en-1-ol); dihydro myrcenol (2,6-dimethyloct-7-en-2-ol); Ebanol™ ((E)-3-methyl-5-(2,2,3-trimethylcyclopent-3-en-1-yl)pent-4-en-2-ol); eugenol (4-allyl-2-methoxyphenol); ethyl linalool ((E)-3,7-dimethylnona-1,6-dien-3-ol); farnesol ((2E,6Z)-3,7,11-trimethyldodeca-2,6,10-trien-1-ol); geraniol ((E)-3,7-dimethylocta-2,6-dien-1-ol); Super Muguet™ ((E)-6-ethyl-3-methyloct-6-en-1-ol); linalool (3,7-dimethylocta-1,6-dien-3-ol); menthol (2-isopropyl-5-methylcyclohexanol); Nerol (3,7-dimethyl-2,6-octadien-1-ol); phenyl ethyl alcohol (2-phenylethanol); Rhodinol™ (3,7-dimethyloct-6-en-1-ol); Sandalore™ (3-methyl-5-(2,2,3-trimethylcyclopent-3-en-1-yl)pentan-2-ol); terpineol (2-(4-methylcyclohex-3-en-1-yl)propan-2-ol); or Timberol™ (1-(2,2,6-trimethylcyclohexyl)hexan-3-ol); 2,4,7-trimethylocta-2,6-dien-1-ol, and/or [1-methyl-2(5-methylhex-4-en-2-yl)cyclopropyl]-methanol;
Aldehydes and ketones, e.g. anisaldehyde (4-methoxybenzaldehyde); alpha amyl cinnamic aldehyde (2-benzylideneheptanal); Georgywood™ (1-(1,2,8,8-tetramethyl-1,2,3,4,5,6,7,8-octahydronaphthalen-2-yl)ethanone); Hydroxycitronellal (7-hydroxy-3,7-dimethyloctanal); Iso E Super® (1-(2,3,8,8-tetramethyl-1,2,3,4,5,6,7,8-octahydronaphthalen-2-yl)ethanone); Isoraldeine® ((E)-3-methyl-4-(2,6,6-trimethylcyclohex-2-en-1-yl)but-3-en-2-one); Hedione® (methyl 3-oxo-2-pentylcyclopentaneacetate); Nympheal (3-(4-isobutyl-2-methylphenyl)propanal); Mahonial (5,9-dimethyl-9-hydroxy-decen-4-al); maltol; methyl cedryl ketone; methylionone; verbenone; and/or vanillin;
Ether and acetals, e.g. Ambrox® (3a,6,6,9a-tetramethyl-2,4,5,5a,7,8,9,9b-octahydro-1H-benzo[e][1]benzofuran); geranyl methyl ether ((2E)-1-methoxy-3,7-dimethylocta-2,6-diene); rose oxide (4-methyl-2-(2-methylprop-1-en-1-yl)tetrahydro-2H-pyran); and/or Spirambrene® (2′,2′,3,7,7-pentamethylspiro[bicyclo[4.1.0]heptane-2,5′-[1,3]dioxane]);
Esters and lactones, e.g. benzyl acetate; cedryl acetate ((1S,6R,8aR)-1,4,4,6-tetramethyloctahydro-1H-5,8a-methanoazulen-6-yl acetate); γ-decalactone (6-pentyltetrahydro-2H-pyran-2-one); Helvetolide® (2-(1-(3,3-dimethylcyclohexyl)ethoxy)-2-methylpropyl propionate); γ-undecalactone (5-heptyloxolan-2-one); and/or vetiveryl acetate ((4,8-dimethyl-2-propan-2-ylidene-3,3a,4,5,6,8a-hexahydro-1H-azulen-6-yl) acetate
Macrocycles, e.g. Ambrettolide ((Z)-oxacycloheptadec-10-en-2-one); ethylene brassylate (1,4-dioxacycloheptadecane-5,17-dione); and/or Exaltolide® (16-oxacyclohexadecan-1-one); and
Heterocycles, e.g. isobutylquinoline (2-isobutylquinoline).

Overall, the compounds of formula (I) can be used alone, as a mixture thereof, or in combination with other fragrance ingredients and/or precursors thereof. Such other fragrance ingredients are also described, for example, in “Perfume and Flavor Chemicals”, S. Arctander, Ed., Vol. I & II, Allured Publishing Corporation, Carol Stream, USA, 2003 and include fragrance compounds of natural or synthetic origin and essential oils.

In a further aspect, there is provided a consumer product comprising at least one compound of formula (I) and a consumer product base.

For example, the consumer product according to the invention is selected from the group consisting of detergents and cleaning agents, hygiene or care products, preferably in the field of body and hair care, cosmetics and household, preferably from the group consisting of perfume extracts, eau de parfums, eau de toilettes, aftershave lotions, eau de colognes, pre-shave products, splash colognes, perfumed refreshing wipes, acidic, alkaline or neutral detergents, textile fresheners, ironing aids, liquid detergents, powder detergents, laundry pre-treatments, fabric softeners, laundry sheets, washing soaps, washing tablets, dish bar soaps, disinfectants, surface disinfectants, air fresheners, aerosol sprays, waxes and polishes, body care products, hand creams and lotions, foot creams and lotions, depilatory creams and lotions, after-shave creams and lotions, tanning creams and lotions, hair care products (in liquid or solid form), dry shampoo, deodorants, antiperspirants, decorative cosmetic products, candles, lamp oils, incense sticks, insecticides, repellents and fuels.

The consumer product for example is selected from fine fragrance, personal care products (body care products, hair care products, cosmetic products) fabric care products, home care products and air care products. As used herein, “consumer product base” means a composition for use as a consumer product to fulfill specific actions, such as cleaning, softening, and caring or the like.

Personal care products to which the compound of formula (I) can be added include for example all kinds of body care products. Especially interesting products are hair care products, for example shampoos, conditioners and hairsprays, and skin care products, like lotions or creams. Furthermore, the compound of formula (I) may be added to soaps, bath and shower gels and deodorants. The compound of formula (I) can be added to cosmetic products.

Home care products to which the compound of formula (I) can be added include all kinds of detergents, window cleaners, hard surface cleaners, all-purpose cleaners and furniture polishes. Preferably, the products are liquids, e.g. fabric detergent or conditioner compositions.

For example, the compounds of formula (I) can act as fragrance precursors in consumer products which further comprise enzymes.

The compound according to formula (I) may be used in a broad range of perfumed consumer products, e.g. in any field of fine and functional perfumery, such as perfumes, air care products, household products, laundry products, body care products and cosmetics. The compound can be employed in widely varying amounts, depending upon the specific article and on the nature and quantity of other odorant ingredients. The proportion of the formula (I) is typically from 0.0001 to 5 weight % of the article. In one embodiment, the compound of formula (I) may be employed in a fabric softener in an amount from 0.001 to 0.3 weight % (e.g. 0.01 to 0.1 including 0.05 weight %). In another embodiment, the compound of formula (I) may be used in fine perfumery but also in consumer products like shampoo, fabric softener or fabric detergents, in amounts from 0.001 to 30 weight % (e.g. up to about 10 or up to 20 weight %), more preferably between 0.01 and 5 weight %. However, these values are given only by way of example, since the experienced perfumer may also achieve effects or may create novel accords with lower or higher concentrations.

In one embodiment there is provided a consumer product comprising an acceptable amount of the compound of formula (I). For example, the fragranced article may comprise 0.000001 weight % to 90 weight % (including 0.00001 weight %; 0.0001 weight %, 0.001 weight %, 0.01 weight %, 0.05 weight %, 0.1 weight %, 0.5 weight %, 1 weight %, 5 weight %, 8 weight %, 10 weight %, 15 weight %, 20 weight %, 25 weight %, 30 weight %, 50 weight %, 60 weight %, 65 weight %) of the compound of formula (I) based on the total amount of the article.

The compound of formula (I) may be employed in a consumer product base simply by directly mixing the compound of the present invention, or a fragrance composition comprising the compound of formula (I), with the consumer product base, or it may, in an earlier step, be entrapped with an entrapment material, for example, polymers, capsules, microcapsules and nanocapsules, liposomes, film formers, absorbents such as carbon or zeolites, cyclic oligosaccharides and mixtures thereof, and then mixed with the consumer product base. The consumer product base might further contain entrapment material able to release other fragrant compounds.

Thus, the invention additionally provides a method of manufacturing a consumer product, comprising the incorporation of a compound of formula (I) either by directly admixing it to the consumer product base or by admixing a fragrance composition comprising the compound of formula (I), which may then be mixed with a consumer product base, using conventional techniques and methods. Through the addition of an acceptable amount of the compound of the present invention as hereinabove described, the odor notes of an applied consumer product will be improved, conferred, enhanced, or modified.

Thus, the invention furthermore provides in another aspect a method to confer, enhance, improve or modify the hedonic properties of a fragrance composition or a consumer product, which method comprises adding to said composition or consumer product at least one compound of formula (I).

The compounds of formula (I) may be prepared starting from the respective phenolic fragrant compound HX which is a compound of formula (II) and an unsaturated alkylhalide, for example an alkenyl chloride, bromide or iodide.

Alternatively, the phenolic fragrant compound HX of formula (II) can be converted to a compound of formula (I) via a Mitsunobu reaction with an unsaturated alcohol in the presence of triphenylphosphine (PPh₃) and diisopropyl azodicarboxylate (DIAD)

Therefore, in a further aspect of the present invention, there is provided a method of making a compound of formula (I), comprising the steps of:
a) providing a phenolic fragrant compound HX,
b) reacting it with an unsaturated alkylhalide or an unsaturated alcohol.

In one embodiment, the starting materials for the process to prepare the compound of formula (I) is obtained from renewable resources. With such a starting material also the final product is accessible from renewable resources.

In a further aspect, the compounds of the present invention are biodegradable, as demonstrated by the Manometric Respirometry test (OECD guideline for the testing of materials No. 301F, Paris 1992).

The invention is now further described with reference to the following non-limiting examples. These examples are for the purpose of illustration only and it is understood that variations and modifications can be made by one skilled in the art.

### DESCRIPTION OF FIGURES

FIG. 1 shows the comparison of color stability of a precursor according to the invention and a free phenolic fragrant compound HX when used in a liquid detergent.

FIG. 2 shows the comparison of performance over time of a precursor according to the invention and a free phenolic fragrant compound HX when used in a liquid detergent.

FIG. 3 shows the setup of a headspace vial used for analysis of volatiles.

### EXAMPLES

### General:

All reactions were performed under argon using solvents and reagents from commercial suppliers without further purification. Solvents for extraction and chromatography were technical grade and used without further purification. Flash chromatography was performed using commercially available prepacked silica gel cartridges. Unless otherwise noted, a mixture of Heptane:MTBE was used as eluent. NMR spectra were recorded with Bruker AW 400 MHz or Avance III HD 400 MHz instruments. The chemical shifts for ¹H NMR spectra was reported in δ (ppm) referenced to the residual proton signal of the deuterated solvent; coupling constants were expressed in Hertz (Hz). ¹³C NMR spectra were referenced to the carbon signals of the deuterated solvent. The following abbreviations are used: s=singlet, d=doublet, t=triplet, q=quartet, quint.=quintuplet, m=multiplet, dd=double doublet, bs=broad singlet. GC/MS spectral data were obtained from an Agilent 6890 N and MSD 5975 using a column HP-5 MS, 30 m, 0.25 mm, 0.25 μm.

### Example 1: (Z)-3-ethoxy-4-(hex-3-en-1-yloxy)benzaldehyde

To a solution of 3-ethoxy-4-hydroxybenzaldehyde (20.0 g, 120 mmol) and potassium carbonate (25.0 g, 180 mmol) in DMF (200 mL) was added (Z)-1-bromohex-3-ene (25.5 g, 156 mmol) dropwise at r.t. and then stirred at 90° C. for 16 hours under argon atmosphere. The reaction conversion was monitored by TLC and GC. After cooling down the reaction solution to r.t., water (150 mL) and MTBE (150 mL) was added. The mixture was extracted with MTBE (2×100 mL), the organic layers were combined and washed with water (3×100 mL), and dried with MgSO₄ and the solvent was removed to give yellow oil. It was then purified by silica gel column chromatography (hexane:MTBE=94:6) to get (Z)-3-ethoxy-4-(hex-3-en-1-yloxy)benzaldehyde (24.5 g, 82% yield) as a colorless oil.

¹H NMR (400 MHz, CDCl₃) δ 9.73 (s, 1H), 7.48-7.24 (m, 2H), 6.86 (d, J=8.1 Hz, 1H), 5.52-5.40 (m, 1H), 5.38-5.26 (m, 1H), 4.12-3.89 (m, 4H), 2.52 (q, J=7.0 Hz, 2H), 2.13-1.89 (m, 2H), 1.36 (t, J=7.0 Hz, 3H), 0.90 (t, J=7.6 Hz, 3H) ppm. ¹³C NMR (101 MHz, CDCl₃) δ 190.9 (d), 154.3 (s), 149.1 (s), 134.8 (d), 130.0 (s), 126.5 (d), 123.4 (d), 111.7 (d), 110.8 (d), 68.5 (t), 64.5 (t), 27.1 (t), 20.6 (t), 14.6 (q), 14.2 (q) ppm. GC/MS (EI): m/z (%): 248 (15) [M⁺], 219 (1), 166 (100), 149 (10), 138 (70), 109 (11), 83 (25), 67 (18). Odor description (1% solution in EtOH on paper blotter, 24 h): powdery (vanillin) creamy

### Example 2: (E)-3-ethoxy-4-(hex-3-en-1-yloxy)benzaldehyde

The compound was obtained from 3-ethoxy-4-hydroxybenzaldehyde (5.0 g, 30.1 mmol, 1.0 equiv) and (E)-1-bromohex-3-ene (6.4 g, 39.1 mmol, 1.3 equiv) according to the procedure of example 1 as light yellow liquid (22.6 mmol, 5.6 g, 75% yield).

¹H NMR (400 MHz, CDCl₃) δ 9.74 (s, 1H), 7.49-7.27 (m, 2H), 6.88 (d, J=8.1 Hz, 1H), 5.63-5.48 (m, 1H), 5.44-5.27 (m, 1H), 4.03 (m, 4H), 2.47 (q, J=6.9 Hz, 2H), 2.11-1.84 (m, 2H), 1.38 (t, J=7.0 Hz, 3H), 0.90 (t, J=7.5 Hz, 3H) ppm. ¹³C NMR (101 MHz, CDCl₃) δ 190.9 (d), 154.4 (s), 149.2 (s), 135.4 (d), 130.0 (s), 126.6 (d), 123.8 (d), 111.9 (d), 111.1 (d), 68.9 (t), 64.6 (t), 32.2 (t), 25.6 (t), 14.7 (q), 13.7 (q) ppm. GC/MS (EI): m/z (%): 248 (12) [M⁺], 233 (1), 166 (75), 149 (7), 138 (43), 83 (32), 55 (100).

Odor description (1% solution in EtOH on paper blotter, 24 h): powdery (vanilla, creamy) slightly fruity (raspberry, strawberry)

### Example 3: 3-ethoxy-4-(hex-3-en-1-yloxy)benzaldehyde

The compound was obtained from 3-ethoxy-4-hydroxybenzaldehyde (20.0 g, 120.3 mmol, 1.0 equiv) and 1-bromohex-3-ene (23.6 g, 144.4 mmol, 1.2 equiv) according to the procedure of example 1 as colorless liquid (89.0 mmol, 22.1 g, 74% yield, E/Z 3:1).

¹H NMR (400 MHz, CDCl₃, mixture of E/Z isomers) δ 9.75 (s, 1H), 7.38-7.26 (m, 2H), 6.88 (d, J=8.1 Hz, 1H), 5.63-5.25 (m, 2H), 4.15-3.92 (m, 4H), 2.57-1.92 (m, 4H), 1.96 (s, 1H), 1.38 (t, J=7.0 Hz, 3H), 0.93-0.88 (m, 3H) ppm. ¹³C NMR (101 MHz, CDCl₃, mixture of E/Z isomers) δ 190.9 (d), 154.3 (s), 149.2 (s), 135.4 (d), 134.9 (d), 130.0 (s), 126.6 (d), 123.7 (d), 123.4 (d), 111.9 (d), 111.7 (d), 111.1 (d), 110.8 (d), 68.9 (t), 68.5 (t), 64.6 (t), 64.5 (t), 32.2 (t), 27.2 (t), 25.7 (t), 20.7 (t), 14.7 (q), 14.3 (q), 13.7 (q) ppm. GC/MS (EI): m/z (%): 248 (18) [M⁺], 166 (100), 149 (9), 138 (67), 83 (26), 67 (14), 55 (89).

Odor description (1% solution in EtOH on paper blotter, 24 h): powdery sweet (vanilla)

### Example 4: (Z)-3-ethoxy-4-(hept-4-en-2-yloxy)benzaldehyde

The compound was obtained from 3-ethoxy-4-hydroxybenzaldehyde (10.0 g, 60.2 mmol, 1.0 equiv) and (Z)-6-bromohept-3-ene (10.66 g, 60.2 mmol, 1.1 equiv) according to the procedure of example 1 as colorless liquid (8.0 mmol, 2.1 g, 13% yield).

¹H NMR (400 MHz, CDCl₃) δ59.83 (s, 1H), 7.51-7.34 (m, 2H), 6.96 (d, J=7.0 Hz, 1H), 5.64-5.38 (m, 2H), 4.52-4.47 (m, 1H), 4.13 (q, J=7.0 Hz, 2H), 2.64-2.28 (m, 2H), 2.19-1.99 (m, 2H), 1.45 (t, J=7.0 Hz, 3H), 1.38 (d, J=6.1 Hz, 3H), 0.96 (t, J=7.5 Hz, 3H) ppm. ¹³C NMR (101 MHz, CDCl₃) δ 190.9 (d), 153.6 (s), 150.0 (s), 134.5 (d), 133.3 (d), 130.1 (s), 126.3 (d), 123.9 (d), 114.3 (d), 111.6 (d), 75.4 (d), 64.6 (t), 33.9 (t), 20.7 (t), 19.4 (q), 14.7 (q), 14.2 (q) ppm. GC/MS (EI): m/z (%): 262 (7) [M⁺], 233 (1), 166 (61), 149 (8), 138 (47), 121 (4), 109 (10), 97 (33), 81 (32), 55 (100).

Odor description (1% solution in EtOH on paper blotter, 24 h): sweet (brown sugar) powdery (vanillin, chocolate, ice cream)

### Example 5: (Z)-4-(4-(hept-4-en-2-yloxy)phenyl)butan-2-one

The compound was obtained from 4-(4-hydroxyphenyl)butan-2-one (4.0 g, 24.4 mmol, 1.0 equiv) and (Z)-6-bromohept-3-ene (6.5 g, 36.5 mmol, 1.2 equiv) according to the procedure of example 1 as colorless liquid (2.7 mmol, 0.7 g, 7% yield).

¹H NMR (400 MHz, CDCl₃) δ 6.99 (d, J=8.5 Hz, 2H), 6.73 (d, J=8.5 Hz, 2H), 5.43-5.30 (m, 2H), 4.29-4.20 (m, 1H), 2.79-2.62 (m, 4H), 2.42-1.95 (m, 7H), 1.35-0.97 (m, 3H), 0.96-0.72 (m, 3H) ppm. ¹³C NMR (101 MHz, CDCl₃) δ 208.2 (s), 156.4 (s), 134.2 (d), 132.9 (s), 129.2 (d), 124.1 (d), 116.0 (d), 73.7 (d), 45.5 (t), 33.9 (t), 30.1 (q), 28.9 (t), 20.8 (t), 19.5 (q), 14.2 (q) ppm. GC/MS (EI): m/z (%): 260 (16) [M⁺], 191 (25), 164 (77), 133 (14), 107 (100), 94 (25), 55 (82).

Odor description (1% solution in EtOH on paper blotter, 24 h): raspberry (fruity, ripe, fresh)

### Example 6: (Z)-4-(4-(hex-3-en-1-yloxy)phenyl)butan-2-one

The compound was obtained from 4-(4-hydroxyphenyl)butan-2-one (20.0 g, 121.8 mmol, 1.0 equiv) and (Z)-1-bromohex-3-ene (23.8 g, 146.1 mmol, 1.2 equiv) according to the procedure of example 1 as colorless liquid (67.4 mmol, 16.6 g, 55% yield).

¹H NMR (400 MHz, CDCl₃) δ 7.00 (d, J=8.3 Hz, 2H), 6.73 (d, J=8.3 Hz, 2H), 5.50-5.40 (m, 1H), 5.38-5.29 (m, 1H), 3.84 (t, J=6.9 Hz, 2H), 2.75 (t, J=7.5 Hz, 2H), 2.64 (t, J=7.5 Hz, 2H), 2.44 (q, J=6.9 Hz, 2H), 2.04 (s, 3H), 2.03-1.96 (m, 2H), 0.91 (t, J=7.5 Hz, 3H) ppm. ¹³C NMR (101 MHz, CDCl₃) δ 208.2 (s), 157.4 (s), 134.3 (d), 133.0 (s), 129.2 (d), 124.1 (d), 114.6 (d), 67.6 (t), 45.5 (t), 30.1 (q), 28.9 (t), 27.4 (t), 20.7 (t), 14.3 (q) ppm. GC/MS (EI): m/z (%): 246 (20) [M⁺], 190 (2), 164 (60), 149 (13), 121 (21), 107 (100), 94 (23), 77 (19), 55 (68).

Odor description (1% solution in EtOH on paper blotter, 24 h): raspberry

### Example 7: (E)-4-(4-(hex-3-en-1-yloxy)phenyl)butan-2-one

The compound was obtained from 4-(4-hydroxyphenyl)butan-2-one (5.0 g, 30.4 mmol, 1.0 equiv) and (E)-1-bromohex-3-ene (6.0 g, 36.5 mmol, 1.2 equiv) according to the procedure of example 1 as colorless liquid (16.2 mmol, 4.0 g, 53% yield).

¹H NMR (400 MHz, CDCl₃) δ 7.00 (d, J=8.2 Hz, 2H), 6.73 (d, J=8.2 Hz, 2H), 5.61-5.46 (m, 1H), 5.45-5.31 (m, 1H), 3.85 (t, J=6.9 Hz, 2H), 2.75 (t, J=7.4 Hz, 2H), 2.63 (t, J=7.4 Hz, 2H), 2.37 (q, J=6.9 Hz, 2H), 2.04 (s, 3H), 1.99-1.88 (m, 2H), 0.90 (t, J=7.5 Hz, 3H) ppm. ¹³C NMR (101 MHz, CDCl₃) δ 208.2 (s), 157.4 (s), 134.8 (d), 132.9 (s), 129.2 (d), 124.5 (d), 114.6 (d), 67.9 (t), 45.5 (t), 32.6 (t), 30.1 (q), 28.9 (t), 25.7 (t), 13.8 (q) ppm. GC/MS (EI): m/z (%): 246 (20) [M⁺], 164 (69), 121 (25), 107 (92), 94 (24), 77 (24), 55 (100).

Odor description (1% solution in EtOH on paper blotter, 24 h): raspberry

### Example 8: (Z)-4-(hex-3-en-1-yloxy)-3-methoxybenzaldehyde

The compound was obtained from 4-hydroxy-3-methoxybenzaldehyde (4.0 g, 26.3 mmol, 1.0 equiv) and (Z)-1-bromohex-3-ene (6.43 g, 39.4 mmol, 1.5 equiv) according to the procedure of example 1 as colorless liquid (21.3 mmol, 5.0 g, 81% yield).

¹H NMR (400 MHz, CDCl₃) δ 9.84 (s, 1H), 7.54-7.35 (m, 2H), 6.97 (d, J=8.2 Hz, 1H), 5.75-5.52 (m, 1H), 5.47-5.26 (m, 1H), 4.09 (t, J=7.2 Hz, 2H), 3.92 (s, 3H), 2.64 (q, J=7.2 Hz, 2H), 2.23-1.92 (m, 2H), 0.99 (t, J=7.5 Hz, 3H) ppm. ¹³C NMR (101 MHz, CDCl₃) δ 190.8 (d), 153.9 (s), 149.8 (s), 135.0 (d), 130.0 (s), 126.7 (d), 123.1 (d), 111.4 (d), 109.3 (d), 68.4 (t), 55.9 (q), 27.1 (t), 20.7 (t), 14.2 (q) ppm. GC/MS (EI): m/z (%): 234 (10) [M⁺], 152 (100), 137 (5), 109 (5), 83 (22), 67 (15), 55 (75).

Odor description (1% solution in EtOH on paper blotter, 24 h): powdery (vanilla) slightly green

### Example 9: methyl (Z)-4-(hex-3-en-1-yloxy)-2-hydroxy-3,6-dimethylbenzoate

The compound was obtained from methyl 2,4-dihydroxy-3,6-dimethylbenzoate (4.0 g, 20.4 mmol, 1.0 equiv) and (Z)-1-bromohex-3-ene (4.0 g, 24.5 mmol, 3.0 equiv) according to the procedure of example 1 as colorless liquid (7.2 mmol, 2.0 g, 35% yield).

¹H NMR (400 MHz, CDCl₃) δ 11.81 (s, 1H), 6.23 (s, 1H), 5.59-5.47 (m, 1H), 5.46-5.34 (m, 1H), 3.97 (t, J=6.7 Hz, 2H), 3.89 (s, 3H), 2.58-2.50 (m, 2H), 2.48 (s, 3H), 2.16-1.98 (m, 5H), 0.99 (t, J=7.5 Hz, 3H) ppm. ¹³C NMR (101 MHz, CDCl₃) δ 171.5 (s), 161.2 (s), 159.8 (s), 139.0 (s), 133.4 (d), 123.0 (d), 110.0 (s), 105.7 (d), 104.2 (s), 66.6 (t), 50.7 (d), 26.4 (t), 23.6 (q), 19.6 (t), 13.2 (q), 6.8 (q) ppm. GC/MS (EI): m/z (%): 278 (17) [M⁺], 231(15), 196 (58), 164 (100), 136 (44), 83 (17), 67 (14), 55 (43).

Odor description (1% solution in EtOH on paper blotter, 24 h): powdery mossy (Evernyl)

### Example 10: (Z)-4-allyl-1-(hex-3-en-1-yloxy)-2-methoxybenzene

The compound was obtained from 4-allyl-2-methoxyphenol (4.0 g, 24.4 mmol, 1.0 equiv) and (Z)-1-bromohex-3-ene (5.2 g, 31.7 mmol, 1.3 equiv) according to the procedure of example 1 as light yellow liquid (17.0 mmol, 4.2 g, 70% yield).

¹H NMR (400 MHz, CDCl₃) δ 6.72 (d, J=8.4 Hz, 1H), 6.66-6.56 (m, 2H), 5.99-5.76 (m, 1H), 5.50-5.40 (m, 1H), 5.38-5.26 (m, 1H), 5.06-4.88 (m, 2H), 3.90 (t, J=7.4 Hz, 2H), 3.76 (s, 3H), 3.24 (d, J=6.7 Hz, 2H), 2.50 (q, J=7.2 Hz, 2H), 2.04-1.97 (m, 2H), 0.89 (t, J=7.5 Hz, 3H) ppm. ¹³C NMR (101 MHz, CDCl₃) δ 148.4 (s), 145.7 (s), 136.7 (d), 133.4 (d), 131.8 (s), 122.8 (d), 119.4 (d), 114.5 (t), 112.2 (d), 111.4 (d), 67.5 (t), 54.9 (q), 38.8 (t), 26.4 (t), 19.6 (t), 13.2 (q) ppm. GC/MS (EI): m/z (%): 246 (15) [M⁺], 164 (100), 149 (25), 131 (16), 91 (21), 55 (35).

Odor description (1% solution in EtOH on paper blotter, 24 h): slightly spicy

### Example 11: (Z)-4-allyl-1-(hex-3-en-1-yloxy)-2-methoxybenzene

The compound was obtained from (E)-2-methoxy-4-(prop-1-en-1-yl)phenol (4.0 g, 24.4 mmol, 1.0 equiv) and (Z)-1-bromohex-3-ene (5.2 g, 31.7 mmol, 1.3 equiv) according to the procedure of example 1 as light yellow liquid (17.5 mmol, 4.3 g, 71% yield).

¹H NMR (400 MHz, CDCl₃) δ 6.92-6.75 (m, 3H), 6.33 (d, J=15.7 Hz, 1H), 6.16-6.01 (m, 1H), 5.58-5.48 (m, 1H), 5.47-5.33 (m, 1H), 3.98 (t, J=7.5 Hz, 2H), 3.86 (s, 3H), 2.58 (q, J=7.2 Hz, 2H), 2.18-2.01 (m, 2H), 1.86 (d, J=6.6 Hz, 3H), 0.97 (t, J=7.5 Hz, 3H) ppm. ¹³C NMR (101 MHz, CDCl₃) δ 149.4 (s), 147.5 (s), 134.5 (d), 131.3 (s), 130.7 (d), 123.8 (d), 118.7 (d), 113.1 (d), 109.1 (d), 68.5 (t), 55.9 (q), 27.4 (t), 20.7 (t), 18.4 (q), 14.3 (q) ppm. GC/MS (EI): m/z (%): 246 (10) [M⁺], 164 (100), 149 (17), 115 (8), 103 (14), 91 (20), 55 (27).

Odor description (1% solution in EtOH on paper blotter, 24 h): slightly spicy woody

### Example 12: 4-(but-3-en-1-yloxy)-3-ethoxybenzaldehyde

The compound was obtained from 3-ethoxy-4-hydroxybenzaldehyde (5.0 g, 30.1 mmol, 1.0 equiv) and 4-bromobut-1-ene (5.3 g, 39.1 mmol, 1.3 equiv) according to the procedure of example 1 as colorless liquid (20.9 mmol, 4.6 g, 70% yield).

¹H NMR (400 MHz, CDCl₃) δ 9.83 (s, 1H), 7.55-7.34 (m, 2H), 6.97 (d, J=8.1 Hz, 1H), 6.08-5.80 (m, 1H), 5.31-5.00 (m, 2H), 4.23-4.01 (m, 4H), 2.63 (q, J=6.8 Hz, 2H), 1.46 (t, J=7.0 Hz, 3H) ppm. ¹³C NMR (101 MHz, CDCl₃) δ 190.9 (d), 154.2 (s), 149.2 (s), 133.8 (d), 130.1 (s), 126.5 (d), 117.5 (t), 112.0 (d), 111.1 (d), 68.3 (t), 64.6 (t), 33.4 (t), 14.7 (q) ppm. GC/MS (EI): m/z (%): 220 (97) [M⁺], 192 (10), 166 (47), 137 (100), 109 (21), 81 (25), 55 (94).

Odor description (1% solution in EtOH on paper blotter, 24 h): powdery (vanilla, creamy, dough)

### Example 13: 4-allyl-1-(but-3-en-1-yloxy)-2-methoxybenzene

The compound was obtained from 4-allyl-2-methoxyphenol (2.5 g, 15.2 mmol, 1.0 equiv) and 4-bromobut-1-ene (2.7 g, 19.8 mmol, 1.3 equiv) according to the procedure of example 1 as light yellow liquid (9.6 mmol, 2.1 g, 62% yield).

¹H NMR (400 MHz, CDCl₃) δ 6.74 (d, J=7.9 Hz, 1H), 6.67-6.54 (m, 2H), 6.02-5.67 (m, 2H), 5.13-5.05 (m, 1H), 5.04-4.94 (m, 3H), 3.96 (t, J=6.9 Hz, 2H), 3.77 (s, 3H), 3.25 (d, J=6.4 Hz, 2H), 2.51 (q, J=6.4 Hz, 2H) ppm. ¹³C NMR (101 MHz, CDCl₃) δ 148.5 (s), 145.6 (s), 136.6 (d), 133.3 (d), 132.0 (s), 119.5 (d), 116.0 (s), 114.6 (s), 112.6 (d), 111.5 (d), 67.5 (t), 54.9 (q), 38.8 (t), 32.7 (t) ppm. GC/MS (EI): m/z (%): 218 (95) [M⁺], 203 (1), 164 (100), 149 (43), 131 (29), 103 (41), 91 (44), 77 (26), 55 (49).

Odor description (1% solution in EtOH on paper blotter, 24 h): slightly spicy (iso eugenol) fruity (berry)

### Example 14: methyl 4-(but-3-en-1-yloxy)-2-hydroxy-3,6-dimethylbenzoate

The compound was obtained from methyl 2,4-dihydroxy-3,6-dimethylbenzoate (10.0 g, 51.0 mmol, 1.0 equiv) and 4-bromobut-1-ene (20.6 g, 152.9 mmol, 3.0 equiv) according to the procedure of example 1 as white solid (24.8 mmol, 6.2 g, 49% yield).

¹H NMR (400 MHz, CDCl₃) δ 11.75 (s, 1H), 6.14 (s, 1H), 5.91-5.63 (m, 1H), 5.32-4.83 (m, 2H), 3.94 (t, J=6.4 Hz, 2H), 3.81 (s, 3H), 2.52-2.42 (m, 2H), 2.40 (s, 3H), 1.98 (s, 3H) ppm. ¹³C NMR (101 MHz, CDCl₃) δ 172.6 (s), 162.2 (s), 160.8 (s), 140.0 (s), 134.3 (d), 117.2 (t), 111.1 (s), 106.8 (d), 105.4 (s), 67.3 (t), 51.7 (q), 33.7 (t), 24.6 (q), 7.8 (q) ppm. GC/MS (EI): m/z (%): 250 (43) [M⁺], 218 (42), 196 (44), 164 (100), 136 (78), 107 (19), 91 (17), 77 (29), 55 (54).

Odor description (1% solution in EtOH on paper blotter, 24 h): slightly mossy

### Example 15: 3-ethoxy-4-((3-methylbut-3-en-1-yl)oxy)benzaldehyde

The compound was obtained from 3-ethoxy-4-hydroxybenzaldehyde (4.0 g, 24.1 mmol, 1.0 equiv) and 4-bromo-2-methylbut-1-ene (4.3 g, 28.9 mmol, 1.2 equiv) according to the procedure of example 1 as colorless liquid (16.6 mmol, 3.9 g, 69% yield).

¹H NMR (400 MHz, CDCl₃) δ 9.73 (s, 1H), 7.48-7.25 (m, 2H), 6.88 (d, J=8.2 Hz, 1H), 4.75 (d, J=15.2 Hz, 2H), 4.10 (t, J=7.0 Hz, 2H), 4.03 (q, J=7.0 Hz, 2H), 2.49 (t, J=7.0 Hz, 2H), 1.74 (s, 3H), 1.36 (t, J=7.0 Hz, 3H) ppm. ¹³C NMR (101 MHz, CDCl₃) δ 190.9 (d), 154.3 (s), 149.2 (s), 141.9 (s), 130.1 (s), 126.5 (d), 112.4 (t), 111.9 (d), 111.0 (d), 67.9 (t), 64.6 (t), 36.9 (t), 22.9 (q), 14.6 (q) ppm. GC/MS (EI): m/z (%): 234 (25) [M⁺], 191 (1), 166 (100), 149 (8), 138 (72), 109 (10), 81 (14), 69 (52).

Odor description (1% solution in EtOH on paper blotter, 24 h): mild, powdery, spicy

### Example 16: 4-(4-((3-methylbut-3-en-1-yl)oxy)phenyl)butan-2-one

The compound was obtained from 4-(4-hydroxyphenyl)butan-2-one (4.0 g, 24.4 mmol, 1.0 equiv) and 4-bromo-2-methylbut-1-ene (4.4 g, 29.2 mmol, 1.2 equiv) according to the procedure of example 1 as colorless liquid (11.2 mmol, 2.6 g, 46% yield).

¹H NMR (400 MHz, CDCl₃) δ 7.00 (d, J=8.3 Hz, 2H), 6.74 (d, J=8.3 Hz, 2H), 4.82-4.62 (m, 2H), 3.96 (t, J=6.8 Hz, 2H), 2.75 (t, J=7.5 Hz, 2H), 2.64 (t, J=7.5 Hz, 2H), 2.40 (t, J=6.8 Hz, 2H), 2.04 (s, 3H), 1.72 (s, 3H) ppm. ¹³C NMR (101 MHz, CDCl₃) δ 208.2 (s), 157.3 (s), 142.3 (s), 133.0 (s), 129.2 (d), 114.6 (d), 111.9 (t), 66.5 (t), 45.4 (t), 37.2 (t), 30.1 (q), 28.9 (t), 22.8 (q) ppm. GC/MS (EI): m/z (%): 232 (34) [M⁺], 164 (50), 149 (12), 121 (20), 107 (100), 94 (21), 77 (16), 69 (23).

Odor description (1% solution in EtOH on paper blotter, 24 h): fruity (raspberry) sweet

### Example 17: (Z)-3-ethoxy-4-(oct-3-en-1-yloxy)benzaldehyde

The compound was obtained from 3-ethoxy-4-hydroxybenzaldehyde (5.0 g, 30.1 mmol, 1.0 equiv) and (Z)-1-bromooct-3-ene (5.75 g, 30.1 mmol, 1.1 equiv) according to the procedure of example 1 as colorless liquid (17.4 mmol, 4.8 g, 58% yield).

¹H NMR (400 MHz, CDCl₃) δ 9.74 (s, 1H), 7.42-7.24 (m, 2H), 6.87 (d, J=8.1 Hz, 1H), 5.51-5.42 (m, 1H), 5.41-5.29 (m, 1H), 4.05 (q, J=7.0 Hz, 2H), 3.99 (t, J=7.1 Hz, 2H), 2.54 (q, J=7.1 Hz, 2H), 2.16-1.93 (m, 2H), 1.38 (t, J=7.0 Hz, 3H), 1.31-1.14 (m, 4H), 0.81 (t, J=7.0 Hz, 3H) ppm. ¹³C NMR (101 MHz, CDCl₃) δ 190.9 (d), 154.3 (s), 149.1 (s), 133.2 (d), 130.0 (s), 126.5 (d), 123.9 (d), 111.8 (d), 110.8 (d), 68.5 (t), 64.5 (t), 31.8 (t), 27.2 (t), 27.1 (t), 22.3 (t), 14.6 (t), 14.0 (t) ppm. GC/MS (EI): m/z (%): 276 (12) [M⁺], 166 (99), 138 (61), 110 (11), 81 (17), 69 (100), 55 (61).

Odor description (1% solution in EtOH on paper blotter, 24 h): vanilla (powdery, spicy, creamy)

### Example 18: 4-(4-((4-methylpent-3-en-1-yl)oxy)phenyl)butan-2-one

The compound was obtained from 4-(4-hydroxyphenyl)butan-2-one (3.02 g, 18.40 mmol, 1.5 equiv) and 5-bromo-2-methylpent-2-ene (2.0 g, 12.27 mmol, 1.0 equiv) according to the procedure of example 1 as colorless liquid (9.7 mmol, 2.4 g, 79% yield).

¹H NMR (400 MHz, CDCl₃) δ 7.00 (d, J=8.5 Hz, 2H), 6.73 (d, J=8.6 Hz, 2H), 5.12 (t, J=7.1 Hz, 1H), 3.81 (t, J=7.1 Hz, 2H), 2.75 (t, J=7.4 Hz, 2H), 2.63 (t, J=7.4 Hz, 2H), 2.38 (q, J=7.0 Hz, 2H), 2.04 (s, 3H), 1.64 (s, 3H), 1.57 (s, 3H) ppm. ¹³C NMR (101 MHz, CDCl₃) δ 207.1 (s), 156.4 (s), 133.3 (s), 131.8 (s), 128.1 (d), 118.6 (d), 113.5 (d), 66.6 (t), 44.4 (t), 29.1 (q), 27.9 (t), 27.3 (t), 24.7 (q), 16.8 (q) ppm. GC/MS (EI): m/z (%): 246 (14) [M⁺], 164 (84), 121 (13), 107 (53), 91 (16), 83 (66), 55 (100).

Odor description (1% solution in EtOH on paper blotter, 24 h): sweet fruity (raspberry ketone)

### Example 19: 3-ethoxy-4-(hept-1-en-4-yloxy)benzaldehyde

To a solution of ethyl vanillin (19.2 g, 116 mmol), hept-1-en-4-ol (15.0 g, 105 mmol) and triphenylphosphane (35.8 g, 137 mmol) in THF (250 mL) was added DIAD (27.6 g, 137 mmol) portionwise at r.t., the temperature was kept below 25° C. during addition. The resulting suspension was stirred for 16 h under argon atmosphere at r.t. The reaction conversion was monitored by TLC and GC. The reaction mixture was filtered, and the solvent was removed by rotary evaporation, the residue was purified by silica gel column chromatography (PE:MTBE=94:6) to give 3-ethoxy-4-(hept-1-en-4-yloxy)benzaldehyde (15.9 g, yield: 58%) as colorless liquid.

¹H NMR (400 MHz, CDCl₃) δ 9.72 (s, 1H), 7.47-7.21 (m, 2H), 6.88 (d, J=8.6 Hz, 1H), 6.08-5.59 (m, 1H), 5.04-4.96 (m, 2H), 4.60-4.20 (m, 1H), 4.01 (q, J=7.0 Hz, 2H), 2.55-2.16 (m, 2H), 1.81-1.50 (m, 2H), 1.46-1.18 (m, 5H), 0.83 (t, J=7.4 Hz, 3H) ppm. ¹³C NMR (101 MHz, CDCl₃) δ 190.8 (d), 154.0 (s), 150.1 (s), 133.8 (d), 130.2 (s), 126.2 (d), 117.6 (t), 114.7 (d), 111.8 (d), 79.0 (d), 64.6 (t), 38.3 (t), 35.8 (t), 18.5 (t), 14.7 (q), 14.0 (q) ppm. GC/MS (EI): m/z (%): 262 (13) [M⁺], 166 (100), 149 (8), 138 (78), 109 (8), 93 (3), 81 (11), 55 (33).

Odor description (1% solution in EtOH on paper blotter, 24 h): powdery (dry, vanilla) fruity (metallic)

### Example 20: 4-(cyclopent-3-en-1-yloxy)-3-ethoxybenzaldehyde

The compound was obtained from 3-ethoxy-4-hydroxybenzaldehyde (25.0 g, 150.4 mmol, 1.0 equiv) and cyclopent-3-en-1-ol (15.19 g, 181 mmol, 1.2 equiv) according to the procedure of example 19 as white solid (116.2 mmol, 27.0 g, 76% yield).

¹H NMR (400 MHz, CDCl₃) δ 9.82 (s, 1H), 7.52-7.32 (m, 2H), 7.07-6.83 (m, 1H), 5.89-5.61 (m, 2H), 5.18-4.88 (m, 1H), 4.10 (q, J=5.0 Hz, 2H), 2.96-2.76 (m, 2H), 2.74-2.54 (m, 2H), 1.43 (t, J=5.0 Hz, 3H) ppm. ¹³C NMR (101 MHz, CDCl₃) δ 190.9 (d), 153.4 (s), 149.6 (s), 129.9 (s), 128.3 (d), 126.4 (d), 113.0 (d), 111.2 (d), 78.1 (d), 64.6 (t), 40.0 (t), 14.6 (q) ppm. GC/MS (EI): m/z (%): 232(23) [M⁺], 166(100), 138(100), 137(78), 109(10), 81(12), 67(61).

Odor description (1% solution in EtOH on paper blotter, 24 h): vanilla (powdery, spicy, creamy)

### Example 21: 4-(4-(hept-1-en-4-yloxy)phenyl)butan-2-one

The compound was obtained from 4-(4-hydroxyphenyl)butan-2-one (17.7 g, 107.9 mmol, 1.1 equiv) and hept-1-en-4-ol (14.0 g, 98.1 mmol, purity: 80%, 1.0 equiv) according to the procedure of example 19 as colorless liquid (67.6 mmol, 17.6 g, 69% yield).

¹H NMR (400 MHz, CDCl₃) δ 6.97 (d, J=8.6 Hz, 2H), 6.72 (d, J=8.6 Hz, 2H), 5.88-5.60 (m, 1H), 5.06-4.91 (m, 2H), 4.23-4.02 (m, 1H), 2.80-2.68 (m, 2H), 2.67-2.55 (m, 2H), 2.41-2.20 (m, 2H), 2.02 (s, 3H), 1.63-1.20 (m, 4H), 0.83 (t, J=7.3 Hz, 3H) ppm. ¹³C NMR (101 MHz, CDCl₃) δ 208.0 (s), 156.8 (s), 134.3 (d), 133.0 (s), 129.3 (d), 117.3 (t), 116.1 (d), 77.2 (d), 45.40 (t), 38.2 (t), 35.9 (t), 30.1 (q), 28.9 (t), 18.7 (t), 14.1 (q) ppm. GC/MS (EI): m/z (%): 260 (23) [M⁺], 219 (16), 177 (3), 164 (73), 149 (14), 121 (20), 107 (100), 94 (30), 77 (10), 55 (25).

Odor description (1% solution in EtOH on paper blotter, 24 h): raspberry

### Example 22: 3-ethoxy-4-(hepta-1,6-dien-4-yloxy)benzaldehyde

The compound was obtained from 3-ethoxy-4-hydroxybenzaldehyde (3.9 g, 23.5 mmol, 1.1 equiv) and hepta-1,6-dien-4-ol (3.0 g, 21.4 mmol, 1.0 equiv) according to the procedure of example 19 as colorless liquid (13.8 mmol, 3.6 g, 65% yield).

¹H NMR (400 MHz, CDCl₃) δ 9.72 (s, 1H), 7.44-7.12 (m, 2H), 6.90 (d, J=8.7 Hz, 1H), 5.98-5.58 (m, 2H), 5.08-4.99 (m, 4H), 4.40-4.28 (m, 1H), 4.06-3.95 (m, 2H), 2.54-1.85 (m, 4H), 1.44-1.12 (m, 3H) ppm. ¹³C NMR (101 MHz, CDCl₃) δ 190.8 (d), 153.6 (s), 150.3 (s), 133.6 (d), 130.5 (s), 126.0 (d), 117.9 (t), 115.4 (d), 111.9 (d), 78.7 (d), 64.6 (t), 37.8 (t), 14.7 (q) ppm. GC/MS (EI): m/z (%): 260 (16) [M⁺], 219 (2), 191 (1), 166 (100), 149 (19), 138 (78), 109 (13), 67 (25).

Odor description (1% solution in EtOH on paper blotter, 24 h): powdery (creamy, vanilla)

### Example 23: 4-(4-(hepta-1,6-dien-4-yloxy)phenyl)butan-2-one

The compound was obtained from 4-(4-hydroxyphenyl)butan-2-one (5.2 g, 31.4 mmol, 1.1 equiv) and hepta-1,6-dien-4-ol (4.0 g, 28.5 mmol, 1.0 equiv) according to the procedure of example 19 as colorless liquid (15.9 mmol, 4.1 g, 56% yield).

¹H NMR (400 MHz, CDCl₃) δ 6.98 (d, J=8.5 Hz, 2H), 6.73 (d, J=8.5 Hz, 2H), 5.86-5.62 (m, 2H), 5.07-4.95 (m, 4H), 4.30-3.98 (m, 1H), 2.73 (t, J=7.5 Hz, 2H), 2.62 (t, J=7.5 Hz, 2H), 2.33 (t, J=6.4 Hz, 4H), 2.03 (s, 3H) ppm. ¹³C NMR (101 MHz, CDCl₃) δ 208.0 (s), 156.5 (s), 134.1 (d), 133.3 (s), 129.3 (d), 117.6 (t), 116.3 (d), 76.9 (d), 45.4 (t), 37.8 (t), 30.1 (q), 28.9 (t) ppm. GC/MS (EI): m/z (%): 258 (25) [M⁺], 217 (15), 164 (64), 149 (64), 121 (21), 107 (100), 94 (29), 77 (16).

Odor description (1% solution in EtOH on paper blotter, 24 h): fruity (raspberry)

### Example 24: 3-ethoxy-4-((6-methylhepta-1,5-dien-4-yl)oxy)benzaldehyde

The compound was obtained from 3-ethoxy-4-hydroxybenzaldehyde (5.8 g, 34.9 mmol, 1.1 equiv) and 6-methylhepta-1,5-dien-4-ol (4.0 g, 31.7 mmol, 1.0 equiv) according to the procedure of example 19 as colorless liquid (16.8 mmol, 4.6 g, 53% yield).

¹H NMR (400 MHz, CDCl₃) δ 9.72 (s, 1H), 7.30-7.26 (m, 2H), 6.80 (d, J=8.0 Hz, 1H), 5.89-3.83 (m, 7H), 2.57-2.31 (m, 2H), 1.62 (s, 3H), 1.61 (s, 3H), 1.39-1.29 (m, 3H) ppm. ¹³C NMR (101 MHz, CDCl₃) δ 190.9 (d), 153.7 (s), 150.0 (s), 136.2 (s), 133.8 (d), 130.2 (s), 126.2 (d), 124.5 (d), 117.6 (t), 115.1 (d), 111.5 (d), 76.5 (d), 64.6 (t), 40.0 (t), 25.6 (q), 18.5 (q), 14.7 (q) ppm. GC/MS (EI): m/z (%): 274 (1) [M⁺], 233 (3), 189 (2), 166 (54), 149 (4), 137 (30), 109 (100), 93 (27), 81 (37), 67 (76), 55 (16).

Odor description (1% solution in EtOH on paper blotter, 24 h): powdery (creamy, vanilla)

### Example 25: 4-(4-(hex-5-en-3-yloxy)phenyl)butan-2-one

The compound was obtained from 4-(4-hydroxyphenyl)butan-2-one (5.4 g, 32.9 mmol, 1.1 equiv) and hex-5-en-3-ol (6.0 g, 30.0 mmol, 1.0 equiv) according to the procedure of example 19 as colorless liquid (12.6 mmol, 3.1 g, 41% yield).

¹H NMR (400 MHz, CDCl₃) δ 6.98 (d, J=8.4 Hz, 2H), 6.73 (d, J=8.4 Hz, 2H), 5.89-5.53 (m, 1H), 5.11-4.67 (m, 2H), 4.23-3.92 (m, 1H), 2.81-2.67 (m, 2H), 2.66-2.52 (m, 2H), 2.38-2.16 (m, 2H), 2.02 (s, 3H), 1.65-1.50 (m, 2H), 0.87 (t, J=7.4 Hz, 3H) ppm. ¹³C NMR (101 MHz, CDCl₃) δ 208.1 (s), 156.8 (s), 134.3 (d), 133.1 (s), 129.2 (d), 117.3 (t), 116.2 (d), 78.6 (d), 45.4 (t), 37.8 (t), 30.1 (q), 28.9 (t), 26.4 (t), 9.6 (q) ppm. GC/MS (EI): m/z (%): 246 (22) [M⁺], 205 (16), 164 (59), 149 (14), 133 (1), 107 (100), 94 (28), 55 (17).

Odor description (1% solution in EtOH on paper blotter, 24 h): sweet (icing sugar cristal) raspberry

### Example 26: 4-(4-(hepta-1,5-dien-4-yloxy)phenyl)butan-2-one

The compound was obtained from 4-(4-hydroxyphenyl)butan-2-one (5.2 g, 31.4 mmol, 1.1 equiv) and (E)-hepta-1,5-dien-4-ol (4.0 g, 28.5 mmol, 1.0 equiv) according to the procedure of example 19 as colorless liquid (17.4 mmol, 4.5 g, 56% yield).

¹H NMR (400 MHz, CDCl₃, mixture of E/Z isomers) δ 6.96 (d, J=8.1 Hz, 2H), 6.72 (d, J=8.1 Hz, 2H), 5.84-5.53 (m, 2H), 5.51-5.31 (m, 1H), 5.12-4.81 (m, 2H), 4.72-4.34 (m, 1H), 2.82-2.54 (m, 5H), 2.50-2.22 (m, 1H), 2.03 (s, 3H), 1.61-1.30 (m, 3H) ppm. ¹³C NMR (101 MHz, CDCl₃, mixture of E/Z isomers) δ 208.1 (s), 156.6 (s), 156.4 (s), 136.3 (d), 134.3 (d), 133.0 (s), 133.0 (d), 132.3 (d), 130.6 (d), 129.7 (d), 129.1 (d), 129.1 (d), 128.3 (d), 117.3 (t), 116.2 (d), 116.2 (d), 115.6 (t), 78.4 (d), 74.4 (d), 45.4 (t), 45.4 (t), 40.2 (t), 36.2 (t), 30.1 (q), 28.9 (t), 21.6 (q), 17.8 (q) ppm. GC/MS (EI): m/z (%): 258 (22) [M⁺], 217 (1), 164 (80), 107 (90), 94 (100), 79 (73), 67 (53), 55 (24).

Odor description (1% solution in EtOH on paper blotter, 24 h): sweet (icing sugar cristal) raspberry

### Example 27: 4-(4-((6-methylhepta-1,5-dien-4-yl)oxy)phenyl)butan-2-one

The compound was obtained from 4-(4-hydroxyphenyl)butan-2-one (6.0 g, 36.5 mmol, 1.0 equiv) and 6-methylhepta-1,5-dien-4-ol (10.1 g, 40.2 mmol, 1.1 equiv) according to the procedure of example 19 as colorless liquid (28.6 mmol, 7.8 g, 78% yield).

¹H NMR (400 MHz, CDCl₃) δ 7.12-6.70 (m, 4H), 5.97-4.67 (m, 5H), 2.87-2.20 (m, 6H), 2.11 (s, 3H), 1.71-1.55 (m, 6H) ppm. ¹³C NMR (101 MHz, CDCl₃) δ 208.0 (s), 156.6 (s), 135.3 (s), 134.2 (d), 132.9 (s), 129.0 (d), 122.6 (d), 117.2 (t), 116.0 (d), 115.5 (t), 74.9 (d), 45.4 (t), 45.3 (t), 40.0 (t), 30.1 (q), 28.9 (t), 25.7 (q), 18.6 (q) ppm. GC/MS (EI): m/z (%): 272 (1) [M⁺], 231 (4), 164 (100), 107 (60), 93 (45), 77 (22), 67 (64).

Odor description (1% solution in EtOH on paper blotter, 24 h): fruity (powdery, sweet, raspberry)

### Example 28: 3-ethoxy-4-((2-methylpent-4-en-2-yl)oxy)benzaldehyde

The compound was obtained from 3-ethoxy-4-hydroxybenzaldehyde (5.0 g, 30.1 mmol, 1.0 equiv) and 2-methylpent-4-en-2-ol (4.9 g, 39.1 mmol, 1.3 equiv) according to the procedure of example 19 as colorless liquid (30.1 mmol, 0.06 g, 1% yield).

¹H NMR (400 MHz, CDCl₃): δ=9.88 (s, 1H), 7.33-7.45 (m, 2H), 7.15 (d, J=8.0 Hz, 1H), 5.95-6.09 (m, 1H), 5.07-5.18 (m, 2H), 4.09 (q, J=6.9 Hz, 2H), 2.51 (br d, J=7.1 Hz, 2H), 1.47 (t, J=6.9 Hz, 3H), 1.34 ppm (s, 6H) ppm. ¹³C NMR (101 MHz, CDCl₃) δ ppm 191.3 (d), 153.9 (s), 151.0 (s), 134.4 (d), 132.5 (s), 124.9 (d), 124.6 (d), 117.9 (t), 111.4 (d), 83.1 (s), 64.2 (t), 47.0 (t), 26.1 (q), 14.7 (q) ppm. GC/MS (EI): m/z (%): 248 (1) [M⁺], 207 (9), 166 (100), 149 (2), 138 (76), 121 (2), 109 (9), 93 (7), 82 (20), 67 (13), 55 (34).

Odor description (1% solution in EtOH on paper blotter, 24 h): powdery (vanilla)

### Example 29: 3-ethoxy-4-((4-methylhepta-3,6-dien-1-yl)oxy)benzaldehyde and 3-ethoxy-4-((4-methylhepta-3,5-dien-1-yl)oxy)benzaldehyde

### a) 7-chloro-4-methylhepta-2,4-diene and 7-chloro-4-methylhepta-1,4-diene

To a solution of 1-cyclopropylethan-1-one (8.00 g, 95 mmol, 1.0 equiv) in diethylether (60 mL) ether was added dropwise at 0° C. allylmagnesium chloride (57 mL, 114 mmol, 1.2 equiv, 2.0M in THF). After having completed the addition, the cooling bath was removed and the reaction mixture was stirred for 1 hour at room temperature. Then it was cooled to 0° C. and a mixture of 50 ml Water and 26 ml conc. sulfuric acid were added dropwise. After stirring for 1 hour, the reaction mixture was added to 100 mL ice cold 2M NaOH and stirred for 20 min. Then it was extracted with 2×120 ml MTBE. The organic layer was washed with 1×120 ml water and 1×120 ml brine. The combined organic layers were dried over MgSO4, filtered and the solvent was evaporated. The crude product was distilled by Kugelrohr distillation (70° C./0.04 mbar) to afford a 2:1 mixture of 7-chloro-4-methylhepta-2,4-diene and 7-chloro-4-methylhepta-1,4-diene (8.79 g, 64% yield, each as E/Z isomers, overall >5 isomers) as a colorless liquid.

¹³C NMR (101 MHz, CDCl₃, mixture of isomers): δ 137.1, 136.5, 136.4, 135.5, 135.4, 134.9, 127.9, 126.7, 124.6, 123.6, 122.8, 121.4, 120.8, 116.0, 115.4, 113.7, 44.4, 44.3, 44.2, 44.0, 44.0, 36.5, 31.6, 31.4, 31.1, 30.8, 27.0, 23.4, 20.6, 18.6, 18.2, 16.2, 12.6.

### b) 3-ethoxy-4-((4-methylhepta-3,6-dien-1-yl)oxy)benzaldehyde and 3-ethoxy-4-((4-methylhepta-3,5-dien-1-yl)oxy)benzaldehyde

The compound was obtained from 3-ethoxy-4-hydroxybenzaldehyde (2.00 g, 12.0 mmol, 1.0 equiv), the mixture of 7-chloro-4-methylhepta-2,4-diene and 7-chloro-4-methylhepta-1,4-diene (2.26 g, 15.7 mmol, 1.3 equiv) described above and 10 mol % of KI according to the procedure of example 1 as a colorless liquid (1.26 g, 38% yield).

¹H NMR (400 MHz, CDCl₃, mixture of isomers): δ 9.83 (s, 1H), 7.47-7.35 (m, 2H), 7.01-6.90 (m, 1H), 6.67-6.51 (m, 0.04H), 6.47 (d, J=14.9 Hz, 0.21H), 6.10 (dd, J=1.0, 15.7 Hz, 0.41H), 5.94-5.87 (m, 0.05H), 5.86-5.71 (m, 0.58H), 5.65 (qd, J=6.6, 15.6 Hz, 0.35H), 5.42 (t, J=7.3 Hz, 0.36H), 5.35-5.19 (m, 0.55H), 5.13-4.97 (m, 0.71H), 4.90 (d, J=14.2 Hz, 0.14H), 4.22-3.91 (m, 4H), 2.95-1.92 (m, 3H), 1.86-1.61 (m, 5H), 1.47 (2 major t, J=7.0 Hz, 3H) ppm. ¹³C NMR (101 MHz, CDCl₃, mixture of isomers): δ 190.9, 154.3, 154.2, 154.2, 149.1, 149.1, 145.1, 137.0, 136.5, 136.5, 135.7, 135.5, 134.9, 132.9, 130.0, 129.9, 129.9, 128.0, 126.5, 126.5, 126.4, 125.2, 123.6, 123.3, 121.8, 120.3, 119.7, 115.9, 115.3, 113.5, 111.8, 111.7, 111.7, 111.7, 110.9, 110.8, 110.8, 110.7, 110.7, 68.5, 68.3, 64.5, 64.5, 64.5, 44.0, 36.4, 28.4, 28.0, 27.9, 27.8, 27.6, 27.2, 23.5, 20.6, 18.6, 18.2, 18.1, 16.2, 14.6, 12.6 ppm.

Odour description (1% solution in EtOH on paper blotter, 24 h): powdery (vanilla) sweet (sugary).

### Example 30: 2-(hex-3-en-1-yloxy)-1-isopropyl-4-methylbenzene

A solution of thymol (2-isopropyl-5-methylphenol) (1.02 g, 6.79 mmol) in acetonitrile (MeCN) (10 mL) at room temperature was treated with K₂CO₃ (1.41 g, 10.18 mmol), tetrabutylammonium iodide (TBAI) (0.376 g, 1.02 mmol) and finally (Z)-1-Bromo-3-hexene (1.33 g, 8.15 mmol). The resulting mixture was heated to reflux for 46 h and then cooled to room temperature and poured into saturated aqueous NH₄Cl solution (50 mL), extracted with methyl tert-butyl ether (MTBE) (2×50 mL), washed with water (20 mL) and brine (20 mL), dried over MgSO₄, filtered and evaporated. The resulting material was purified by chromatography on silica gel eluting with a gradient of MTBE in heptane, followed by Kugelrohr distillation at 130-140° C. at 0.06 mbar to give 2-(hex-3-en-1-yloxy)-1-isopropyl-4-methylbenzene (0.30 g, 18% yield) as a colorless oil.

¹H NMR (400 MHz, CDCl₃, 298 K, mixture of E and Z isomers) δ (ppm)=7.12 (d, J=7.6 Hz, 1H), 6.77 (d, J=7.6 Hz, 1H), 6.69 (s, 1H), 5.71-5.45 (m, 2H), 4.00 (dt, J=2.6, 6.7 Hz, 2H), 3.39-3.26 (m, 1H), 2.63-2.49 (m, 2H), 2.37-2.34 (m, 3H), 2.20-2.02 (m, 2H), 1.24 (d, J=6.8 Hz, 6H), 1.07-1.00 (m, 3H). ¹³C NMR (101 MHz, CDCl₃, 298 K, mixture of E and Z isomers) δ (ppm)=156.1, 136.2, 134.6, 134.1, 134.1, 134.0, 125.8, 125.8, 125.0, 124.6, 121.0, 120.9, 112.3, 112.3, 67.8, 67.6, 32.8, 27.6, 26.7, 26.7, 25.7, 22.9, 22.7, 22.7, 21.4, 20.7, 14.3, 13.8. MS (EI, 70 eV): 232 (10, [M]+*), 217 (1), 150 (28), 135 (100), 83 (14).

Odour description (1% solution in EtOH on paper blotter, 24 h): aromatic, thymol, menthol

### Example 31: 3-ethoxy-4-((6-methylhept-3-en-1-yl)oxy)benzaldehyde

### a) 1-cyclopropyl-3-methylbutan-1-ol

A solution of cyclopropanecarbaldehyde (2.50 g, 35.7 mmol) in THF (tetrahydrofuran) (20 mL) was added slowly to a solution of isobutylmagnesium chloride (2 M in in THF, 19.6 mL, 39.2 mmol) at 5° C. The mixture was then allowed to reach room temperature and stirred for 2 h. The mixture was poured onto sat. aq. NH₄Cl solution (200 mL), extracted with MTBE (2×200 mL), washed with brine (100 mL), dried over MgSO₄, filtered and concentrated to give 1-cyclopropyl-3-methylbutan-1-ol (4.6 g, quantitative yield) as a colorless oil which was used without further purification.

MS (EI, 70 eV): 113 (1, [M]+*-CH_{3*}), 95 (3), 71 (100), 57 (21), 43 (58).

### b) (E)-1-bromo-6-methylhept-3-ene

A solution of 1-cyclopropyl-3-methylbutan-1-ol (4.00 g, 31.2 mmol) in THF (tetrahydrofuran) (20 mL) was slowly added to a mixture of HBr (48% aqueous solution, 4.24 mL, 37.4 mmol) and sodium dodecyl sulfate (0.3 g, 1 mmol) at 5° C. The mixture was diluted with MTBE (100 mL), washed with water (5×100 mL), dried over MgSO₄ and concentrated to give (E)-1-bromo-6-methylhept-3-ene (4.71 g, 93% purity, 73% yield), which was used in the next step without further purification.

¹H NMR (400 MHz, CDCl₃, 298 K) δ (ppm)=5.58-5.47 (m, 1H), 5.42-5.33 (m, 1H), 3.37 (t, J=7.2 Hz, 2H), 2.56 (dq, J=1.0, 7.0 Hz, 2H), 1.90 (dt, J=0.9, 6.9 Hz, 2H), 1.67-1.55 (m, 1H), 0.88 (d, J=6.6 Hz, 6H). ¹³C NMR (101 MHz, CDCl₃, 298 K) δ (ppm)=132.7, 127.5, 41.9, 36.1, 33.0, 28.3, 22.2. MS (EI, 70 eV): 192 (2), 190 (2, [M]+*), 175 (1), 148 (5), 133 (2), 111 (42), 79 (5), 69 (91), 56 (86), 43 (100).

### c) 3-ethoxy-4-((6-methylhept-3-en-1-yl)oxy)benzaldehyde

A mixture of ethyl vanillin (3-ethoxy-4-hydroxybenzaldehyde) (3.90 g, 23.5 mmol), (E)-1-bromo-6-methylhept-3-ene (4.71 g, 24.6 mmol), tetrabutylammonium bromide (378 mg, 1.17 mmol) and K₂CO₃ (3.89 g, 1.2 Eq, 28.2 mmol) in water (20 mL) was heated up to reflux for 5 h. The reaction mixture was cooled and diluted with water (100 mL) and extracted with MTBE (methyl tert-butyl ether) (2×50 mL). The org. layers were washed with water (30 mL) and brine (30 mL), dried over MgSO₄, filtered and concentrated. The resulting material was purified by chromatography on silica gel eluting with a gradient of MTBE in heptane, followed by Kugelrohr distillation under vacuum to give 3-ethoxy-4-((6-methylhept-3-en-1-yl)oxy)benzaldehyde (3.88 g, 60% yield) as a colorless oil.

¹H NMR (400 MHz, CDCl₃, 298 K) δ (ppm)=9.83 (s, 1H), 7.45-7.38 (m, 2H), 6.97 (d, J=8.2 Hz, 1H), 5.63-5.53 (m, 1H), 5.52-5.42 (m, 1H), 4.19-4.07 (m, 4H), 2.62-2.53 (m, 2H), 1.91 (dt, J=0.9, 6.8 Hz, 2H), 1.67-1.56 (m, 1H), 1.47 (t, J=7.0 Hz, 3H), 0.88 (d, J=6.6 Hz, 6H). ¹³C NMR (101 MHz, CDCl₃, 298 K) δ (ppm)=191.0, 154.4, 149.2, 132.6, 130.0, 126.6, 125.9, 111.9, 111.0, 68.8, 64.6, 42.0, 32.3, 28.3, 22.3, 14.7. MS (EI, 70 eV): 276 (4, [M]+*), 166 (53), 138 (32), 69 (100), 55 (48).

Odour description (1% solution in EtOH on paper blotter, 24 h): powdery, vanilla, green, hazelnut, chocolate.

### Example 32: (E)-3-ethoxy-4-((5-methylhex-3-en-1-yl)oxy)benzaldehyde

### a) (E)-1-bromo-5-methylhex-3-ene

A solution of isopropylmagnesium bromide (0.75 molar in THF, 38.8 mL, 29.1 mmol) was cooled to 5° C. and treated slowly with a solution of cyclopropanecarbaldehyde (1.70 g, 24.3 mmol) in THF (10 mL) and the resulting mixture stirred at room temperature for 1 h. The mixture was then treated slowly over 20 min with a solution of H₂SO₄ (16.4 g, 8.89 mL, 167 mmol) in Water (8 mL) and then heated to 50° C. for 4 h. The mixture was cooled and poured into iced 2M NaOH solution (200 mL), extracted with MTBE (2×100 mL), washed with water (100 mL) and brine (50 mL), dried over MgSO₄, filtered and concentrated to give (E)-1-bromo-5-methylhex-3-ene (3.83 g, 89% yield) as a pale yellow oil which was used without further purification.

MS (EI, 70 eV): 178 (4), 176 (4, [M]+*), 161 (1), 133 (1), 97 (27), 93 (4), 83 (4), 69 (100), 55 (41), 41 (47).

### b) (E)-3-ethoxy-4-((5-methylhex-3-en-1-yl)oxy)benzaldehyde

The compound was prepared according to the procedure of example 31c from 3-ethoxy-4-hydroxybenzaldehyde (3.50 g, 21.1 mmol), (E)-1-bromo-5-methylhex-3-ene (3.73 g, 21.1 mmol), tetrabutylammonium bromide (339 mg, 1.05 mmol) and K₂CO₃ (3.49 g, 25.3 mmol) to give (E)-3-ethoxy-4-((5-methylhex-3-en-1-yl)oxy)benzaldehyde (1.83 g, 33% yield) as a colorless oil.

¹H NMR (400 MHz, CDCl₃, 298 K) δ (ppm)=9.83 (s, 1H), 7.45-7.39 (m, 2H), 6.97 (d, J=8.2 Hz, 1H), 5.61-5.52 (m, 1H), 5.48-5.38 (m, 1H), 4.15 (q, J=7.0 Hz, 2H), 4.10 (t, J=7.1 Hz, 2H), 2.55 (q, J=7.0 Hz, 2H), 2.27 (qd, J=6.7, 13.4 Hz, 1H), 1.47 (t, J=7.0 Hz, 3H), 0.98 (d, J=6.7 Hz, 6H). ¹³C NMR (101 MHz, CDCl₃, 298 K) δ (ppm)=191.0, 154.4, 149.2, 141.0, 130.0, 126.6, 121.7, 111.9, 111.1, 68.9, 64.6, 32.2, 31.1, 22.4, 14.7. MS (EI, 70 eV): 262 (5, [M]+*), 166 (40), 138 (33), 96 (38), 55 (100).

Odour description (1% solution in EtOH on paper blotter, 24 h): powdery, vanilla, green, hazelnut, chocolate.

### Example 33: 3-ethoxy-4-(pent-3-en-1-yloxy)benzaldehyde

The compound was obtained from 3-ethoxy-4-hydroxybenzaldehyde (26.5 g, 159 mmol, 1.0 equiv) and 5-bromopent-2-ene (35.0 g, 223 mmol, 1.4 equiv, 95% purity) according to the procedure of example 1 as light yellow liquid (29.6 g, 78% yield, E/Z 93:7), after purification by fractional distillation under high vacuum.

¹H NMR (400 MHz, CDCl₃) for major E isomer: δ 9.77 (s, 1H), 7.42-7.30 (m, 2H), 6.90 (d, J=8.3 Hz, 1H), 5.63-5.50 (m, 1H), 5.50-5.38 (m, 1H), 4.07 (q, J=7.1 Hz, 2H), 4.02 (t, J=7.0 Hz, 2H), 2.54-2.40 (m, 2H), 1.69-1.58 (m, 3H), 1.40 (t, J=7.0 Hz, 3H) ppm.

Characteristic signal for minor Z isomer: ¹H NMR (400 MHz, CDCl₃) δ 2.57 (pseudo q, J=6.8 Hz, 2H) ppm. ¹³C NMR (101 MHz, CDCl₃) for major E isomer: δ 190.6, 154.1, 148.9, 129.8, 128.0, 126.3, 125.8, 111.7, 110.8, 68.6, 64.3, 32.1, 17.8, 14.4 ppm. Characteristic signals for Z isomer: ¹³C NMR (101 MHz, CDCl₃) for minor Z isomer: δ 154.1, 126.9, 124.8, 111.5, 110.6, 68.1, 64.3, 26.7, 12.7 ppm. GC/MS (EI) major E isomer: m/z (%): 234 (5, [M]⁺*), 166 (43), 138 (38), 137 (40), 81 (10), 69 (100), 53 (11), 41 (78), 39 (15), 29 (18), 27 (12).

GC/MS (EI) minor Z isomer: m/z (%): 234 (10, [M]+*), 166 (68), 138 (67), 137 (68), 81 (12), 69 (100), 53 (14), 41 (88), 39 (20), 29 (23), 27 (18).

Odour description (1% solution in EtOH on paper blotter, 24 h): mild, powdery, vanilla, peppery.

### Example 34: 3-ethoxy-4-((4-methylpent-3-en-1-yl)oxy)benzaldehyde

The compound was obtained from 3-ethoxy-4-hydroxybenzaldehyde (17.3 g, 104 mmol, 1.0 equiv) and 5-bromo-2-methylpent-2-ene (22.0 g, 135 mmol, 1.3 equiv) according to the procedure of example 1 as light yellow liquid (21.3 g, 82% yield), after purification by fractional distillation under high vacuum.

¹H NMR (400 MHz, CDCl₃): δ 9.81 (s, 1H), 7.38 (t, J=2.7 Hz, 2H), 6.94 (d, J=8.1 Hz, 1H), 5.20 (tt, J=1.3, 7.3 Hz, 1H), 4.13 (q, J=6.8 Hz, 2H), 4.03 (t, J=7.2 Hz, 2H), 2.55 (q, J=7.1 Hz, 2H), 1.72 (d, J=0.7 Hz, 3H), 1.67 (s, 3H), 1.45 (t, J=7.0 Hz, 3H) ppm. ¹³C NMR (101 MHz, CDCl₃) δ 190.8, 154.3, 149.0, 134.9, 129.8, 126.5, 118.8, 111.6, 110.7, 68.5, 64.4, 27.9, 25.7, 17.8, 14.6 ppm. GC/MS (EI) m/z (%): 248 (2, [M]⁺*), 166 (31), 138 (14), 137 (16), 83 (96), 67 (13), 55 (100), 41 (40), 39 (13), 29 (20), 27 (11).

Odour description (1% solution in EtOH on paper blotter, 24 h): powdery, vanilla, chocolate.

### Example 35: (Z)-2-ethoxy-1-(hex-3-en-1-yloxy)-4-methylbenzene and (Z)-1-ethoxy-2-(hex-3-en-1-yloxy)-4-methylbenzene

The compound mixture was obtained from a 1:1 mixture of 2-ethoxy-4-methylphenol and 2-ethoxy-5-methylphenol (3.00 g, 19.7 mmol, 1.0 equiv, prepared according to EP0179532) and (Z)-1-iodohex-3-ene (6.21 g, 29.6 mmol, 1.5 equiv) according to the procedure of example 1 as colorless liquid (1.68 g, 35% yield, 1:1 mixture of regioisomers).

¹H NMR (400 MHz, CDCl₃, for the 1:1 regioisomer mixture): δ 6.80 (2×d, J=8.1 Hz, 1H), 6.75-6.66 (m, 2H), 5.61-5.50 (m, 1H), 5.49-5.39 (m, 1H), 4.11-4.02 (m, 2H), 4.02-3.95 (2×t, J=7.1 Hz, 2H), 2.65-2.52 (m, 2H), 2.29 (s, 3H), 2.19-2.05 (m, 2H), 1.48-1.39 (m, 3H), 1.05-0.94 (2×t, J=7.6 Hz, 3H) ppm. ¹³C NMR (101 MHz, CDCl₃) δ 148.7, 148.7, 146.6, 146.6, 134.2, 134.2, 130.8, 130.7, 124.1, 124.1, 121.2, 121.0, 115.0, 114.9, 114.2, 114.2, 69.0, 68.6, 64.9, 64.5, 27.5, 27.5, 20.9, 20.9, 20.6, 14.9, 14.9, 14.3 ppm.

Odour description (1% solution in EtOH on paper blotter, 24 h): spicy, eugenol, clove, phenolic, vanilla, medicinal.

### Example 36: 4-(4-((4-methylhex-3-en-1-yl)oxy)phenyl)butan-2-one

The compound was obtained from 4-(4-hydroxyphenyl)butan-2-one (2.00 g, 12.2 mmol, 1.0 equiv) and 1-bromo-4-methylhex-3-ene (2.80 g, 15.8 mmol, 1.3 equiv, prepared according to Zarbin, Paulo H. G.; et al., Journal of Chemical Ecology 2012, 38, 825) according to the procedure of example 1 as colorless liquid (1.12 g, 35% yield, E/Z 86:14).

¹H NMR (400 MHz, CDCl₃, mixture of isomers): δ 7.16-7.01 (m, 2H), 6.90-6.74 (m, 2H), 5.26-5.09 (m, 1H), 3.92 (2×t, J=7.1 Hz, 2H), 2.88-2.79 (m, 2H), 2.78-2.69 (m, 2H), 2.54-2.42 (m, 2H), 2.14 (s, 3H), 2.11-1.96 (m, 2H), 1.75-1.64 (m, 3H), 1.07-0.95 (2×t, J=7.6 Hz, 3H) ppm. ¹³C NMR (101 MHz, CDCl₃, mixture of isomers) δ 208.1, 157.4, 140.0 (Z), 139.8, 132.8, 129.1, 119.1 (Z), 117.9, 114.5, 114.5, 67.8 (Z), 67.7, 45.5, 32.3, 30.1, 28.9, 28.1, 27.9 (Z), 24.9 (Z), 22.9 (Z), 16.1, 12.8, 12.6 ppm.

Odour description (1% solution in EtOH on paper blotter, 24 h): fruity, raspberry, sweet, powdery.

### Example 37: 3-methoxy-4-((4-methylpent-3-en-1-yl)oxy)benzaldehyde

The compound was obtained from 4-hydroxy-3-methoxybenzaldehyde (2.00 g, 13.2 mmol, 1.0 equiv) and 5-bromo-2-methylpent-2-ene (2.79 g, 17.1 mmol, 1.3 equiv) according to the procedure of example 1 as colorless liquid (2.54 g, 82% yield).

¹H NMR (400 MHz, CDCl₃, mixture of isomers): δ 9.84 (s, 1H), 7.44-7.41 (m, 1H), 7.40 (s, 1H), 6.96 (d, J=8.1 Hz, 1H), 5.22-5.17 (m, 1H), 4.06 (t, J=7.5 Hz, 2H), 3.92 (s, 3H), 2.58 (q, J=7.3 Hz, 2H), 1.73 (d, J=1.0 Hz, 3H), 1.66 (s, 3H). ¹³C NMR (101 MHz, CDCl₃, mixture of isomers) δ 190.8, 154.0, 149.8, 135.1, 129.9, 126.7, 118.5, 111.3, 109.2, 68.5, 56.0, 27.9, 25.7, 17.8 ppm.

Odour description (1% solution in EtOH on paper blotter, 24 h): powdery, vanilla, creamy.

### Example 38: 3-methoxy-4-((4-methylhept-3-en-1-yl)oxy)benzaldehyde

The compound was obtained from 4-hydroxy-3-methoxybenzaldehyde (1.32 g, 8.68 mmol, 1.0 equiv) and 1-bromo-4-methylhept-3-ene (2.16 g, 11.3 mmol, 1.3 equiv, prepared according to Zarbin, Paulo H. G.; et al., Journal of Chemical Ecology 2012, 38, 825) according to the procedure of example 1 as light yellow liquid (1.26 g, 55% yield, E/Z 81:19).

¹H NMR (400 MHz, CDCl₃, mixture of isomers): δ 9.85 (s, 1H), 7.46-7.42 (2×d, J=8.1 Hz, 1H), 7.41 (2×s, 1H), 7.00-6.95 (2×d, J=8.1 Hz, 1H), 5.27-5.11 (m, 1H), 4.12-4.02 (m, 2H), 3.93 (s, 3H), 2.68-2.53 (m, 2H), 2.10-1.93 (m, 2H), 1.74-1.61 (m, 3H), 1.49-1.36 (m, 2H), 0.93-0.83 (2×t, J=7.3 Hz, 3H) ppm. ¹³C NMR (101 MHz, CDCl₃, mixture of isomers) δ 190.9, 154.0, 154.0 (Z), 149.8, 149.8 (Z), 139.1 (Z), 138.9, 129.9, 126.7, 118.9 (Z), 118.2, 111.4, 111.3 (Z), 109.3, 109.2 (Z), 68.7 (Z), 68.6, 56.0, 41.8, 33.9 (Z), 27.8, 27.7 (Z), 23.4 (Z), 21.1 (Z), 20.9, 16.0, 13.9 (Z), 13.6 ppm.

Odour description (1% solution in EtOH on paper blotter, 24 h): powdery, vanilla, chocolate.

### Example 39: (Z)-1-ethoxy-2-(hex-3-en-1-yloxy)benzene

The compound was obtained from 2-ethoxyphenol (1.64 g, 11.9 mmol, 1.0 equiv) and (Z)-1-iodohex-3-ene (3.50 g, 16.7 mmol, 1.4 equiv) according to the procedure of example 1 as light yellow liquid (0.78 g, 30% yield).

¹H NMR (400 MHz, CDCl₃): δ 6.91 (s, 4H), 5.61-5.51 (m, 1H), 5.51-5.40 (m, 1H), 4.10 (q, J=6.9 Hz, 2H), 4.02 (t, J=7.2 Hz, 2H), 2.67-2.53 (m, 2H), 2.19-2.05 (m, 2H), 1.46 (t, J=7.0 Hz, 3H), 1.01 (t, J=7.6 Hz, 3H) ppm. ¹³C NMR (101 MHz, CDCl₃) δ 148.9, 148.8, 134.3, 124.0, 121.1, 121.0, 113.9, 113.8, 68.6, 64.5, 27.4, 20.6, 14.9, 14.3 ppm.

Odour description (1% solution in EtOH on paper blotter, 24 h): medicinal, phenolic, vanilla.

### Example 40: 3-ethoxy-4-((4-methylhex-3-en-1-yl)oxy)benzaldehyde

The compound was obtained from 3-ethoxy-4-hydroxybenzaldehyde (2.00 g, 12.2 mmol, 1.0 equiv) and 1-bromo-4-methylhex-3-ene (2.77 g, 15.7 mmol, 1.3 equiv, prepared according to Zarbin, Paulo H. G.; et al., Journal of Chemical Ecology 2012, 38, 825) according to the procedure of example 1 as colorless liquid (1.80 g, 57% yield, E/Z 82:18).

¹H NMR (400 MHz, CDCl₃): 9.83 (s, 1H), 7.44-7.40 (2×d, J=8.1 and 8.3 Hz, 1H), 7.40 (2×s, 1H), 6.99-6.94 (2×d, J=8.3 and 8.1 Hz, 1H), 5.23-5.15 (m, 1H), 4.14 (q, J=6.9 Hz, 2H), 4.10-4.02 (2×t, J=7.3 Hz, 2H), 2.58 (q, J=7.2 Hz, 2H), 2.13-1.98 (m, 2H), 1.75-1.65 (m, 3H), 1.47 (t, J=7.0 Hz, 3H), 1.01 (t, J=7.5 Hz, 3H) ppm. ¹³C NMR (101 MHz, CDCl₃) δ 190.9, 154.4, 154.3 (Z), 149.1, 140.6 (Z), 140.4, 129.9 (Z), 126.5, 118.4 (Z), 117.2, 111.7, 111.7 (Z), 110.9, 110.7 (Z), 68.8 (Z), 68.6, 64.5, 64.5 (Z), 32.3, 27.8, 27.6 (Z), 24.9 (Z), 22.9 (Z), 16.1, 14.6, 12.9 (Z), 12.6 ppm.

Odour description (1% solution in EtOH on paper blotter, 24 h): powdery, vanilla, creamy, chocolate, slightly butyric.

### Example 41: (Z)-1-(hex-3-en-1-yloxy)-3-methoxy-5-methylbenzene

The compound was obtained from 3-methoxy-5-methylphenol (2.0 g, 18.8 mmol, 1.0 equiv) and (Z)-1-iodohex-3-ene (3.95 g, 18.8 mmol, 1.3 equiv) according to the procedure of example 1 as colourless liquid (0.63 g, 20% yield).

¹H NMR (400 MHz, CDCl₃): 6.37-6.33 (m, 2H), 6.33-6.28 (m, 1H), 5.60-5.51 (m, 1H), 5.48-5.40 (m, 1H), 3.94 (t, J=7.0 Hz, 2H), 3.78 (s, 3H), 2.59-2.50 (m, 2H), 2.31 (s, 3H), 2.18-2.06 (m, 2H), 1.01 (t, J=7.6 Hz, 3H) ppm. ¹³C NMR (101 MHz, CDCl₃) δ 160.6, 160.0, 140.1, 134.3, 124.1, 107.6, 107.1, 98.0, 67.4, 55.2, 27.4, 21.8, 20.7, 14.3 ppm.

Odour description (1% solution in EtOH on paper blotter, 24 h): green, bell pepper.

### Example 42: 1-ethyl-4-((4-methylpent-3-en-1-yl)oxy)benzene

The compound was obtained from 4-ethylphenol (2.50 g, 20.5 mmol, 1.0 equiv) and 5-bromo-2-methylpent-2-ene (4.32 g, 22.5 mmol, 1.1 equiv, 85% purity) according to the procedure of example 1 as a colorless liquid (2.34 g, 56% yield).

¹³C NMR (101 MHz, CDCl₃): δ 157.0, 136.3, 134.3, 128.6, 119.7, 114.4, 67.7, 28.3, 28.0, 25.7, 17.8, 15.9 ppm.

Odour description (1% solution in EtOH on paper blotter, 24 h): animalic, leathery, phenolic, paracresol.

### Example 43: 1-methoxy-3-methyl-5-((4-methylpent-3-en-1-yl)oxy)benzene

The compound was obtained from 3-methoxy-5-methylphenol (2.50 g, 18.1 mmol, 1.0 equiv) and 5-bromo-2-methylpent-2-ene (3.82 g, 19.9 mmol, 1.1 equiv, 85% purity) according to the procedure of example 1 as a colorless liquid (2.20 g, 56% yield).

¹³C NMR (101 MHz, CDCl₃): δ 160.6, 160.1, 140.1, 134.3, 119.6, 107.6, 107.1, 98.0, 67.6, 55.2, 28.2, 25.7, 21.8, 17.8 ppm.

Odour description (1% solution in EtOH on paper blotter, 24 h): animalic, leathery, phenolic, paracresol, green, hay, rooty.

### Example 44: 1-methyl-4-((4-methylpent-3-en-1-yl)oxy)benzene

The compound was obtained from p-cresol (3.00 g, 27.7 mmol, 1.0 equiv) and 5-bromo-2-methylpent-2-ene (4.98 g, 30.5 mmol, 1.1 equiv) according to the procedure of example 1 as a colorless liquid (2.58 g, 49% yield).

¹³C NMR (101 MHz, CDCl₃): δ 156.9, 134.3, 129.8, 129.7, 119.7, 114.4, 67.7, 28.3, 25.7, 20.4, 17.8 ppm.

Odour description (1% solution in EtOH on paper blotter, 24 h): animalic, leathery, phenolic, paracresol.

### Example 45: 1-((4-methylpent-3-en-1-yl)oxy)-3-propylbenzene

The compound was obtained from 3-propylphenol (2.50 g, 18.4 mmol, 1.0 equiv) and 5-bromo-2-methylpent-2-ene (3.87 g, 20.2 mmol, 1.1 equiv, 85% purity) according to the procedure of example 1 as a colorless liquid (1.67 g, 42% yield).

¹³C NMR (101 MHz, CDCl₃): δ 159.0, 144.3, 134.3, 129.1, 120.8, 119.7, 114.8, 111.5, 67.5, 38.1, 28.3, 25.7, 24.4, 17.8, 13.8 ppm.

Odour description (1% solution in EtOH on paper blotter, 24 h): animalic, leathery, phenolic, paracresol, green, hay, rooty.

### Example 46: (Z)-1-(2-(hex-3-en-1-yloxy)phenyl)propan-1-one

The compound was obtained from 1-(2-hydroxyphenyl)propan-1-one (8.0 g, 53.3 mmol, 1.0 equiv) and (Z)-1-chlorohex-3-ene (9.5 g, 79.9 mmol, 1.5 equiv) according to the process of example 1 as colorless liquid (21.0 mmol, 5.0 g, 39% yield).

¹H NMR (400 MHz, CDCl₃) δ 7.70-7.66 (m, 1H), 7.46-7.37 (m, 1H), 7.00-6.88 (m, 2H), 5.56-5.41 (m, 2H), 4.06 (t, J=6.7 Hz, 2H), 3.01 (q, J=7.3 Hz, 2H), 2.62-2.57 (m, 2H), 2.14-2.06 (m, 2H), 1.16 (t, J=7.3 Hz, 3H), 0.99 (t, J=7.5 Hz, 3H) ppm. ¹³C NMR (101 MHz, CDCl₃) δ 203.6, 157.9, 134.5, 133.1, 130.3, 128.5, 124.2, 120.6, 112.2, 68.1, 37.2, 27.3, 20.7, 14.2, 8.5 ppm. GC/MS (EI): m/z (%): 232 (1) [M⁺], 203 (5), 185 (5), 163 (11), 150 (27), 121 (100), 82 (26), 55 (38).

Odor description (1% solution in EtOH on paper blotter, 24 h): green, wintergreen

### Example 47: 3-ethoxy-4-(((Z)-hex-3-en-1-yl)oxy)benzaldehyde O-methyl oxime

A mixture of (Z)-3-ethoxy-4-(hex-3-en-1-yloxy)benzaldehyde (1.0 g, 4.0 mmol, 1.0 equiv), O-methylhydroxylamine hydrochloride (0.5 g, 6.0 mmol, 1.5 equiv) and sodium acetate (1.0 g, 12.1 mmol) in Methanol (20 ml) and Water (10 ml) was stirred for 16 hours under argon atmosphere at r.t. The solvent was removed by rotary evaporation, the residue was purified by silica gel column chromatography (PE:MTBE=9:1) and give 3-ethoxy-4-(((Z)-hex-3-en-1-yl)oxy)benzaldehyde O-methyl oxime (4.0 mmol, 1.1 g, yield: 98%) as light yellow liquid.

¹H NMR (400 MHz, CDCl₃) δ 7.89 (s, 1H), 7.14 (s, 1H), 6.90 (d, J=8.2 Hz, 1H), 6.75 (d, J=8.2 Hz, 1H), 5.49-5.31 (m, 2H), 4.03 (q, J=6.9 Hz, 2H), 3.92 (t, J=7.0 Hz, 2H), 3.86 (s, 3H), 2.54-2.46 (m, 2H), 2.08-1.99 (m, 2H), 1.37 (t, J=7.0 Hz, 3H), 0.91 (t, J=7.5 Hz, 3H) ppm. ¹³C NMR (101 MHz, CDCl₃) δ 150.4, 149.0, 148.5, 134.6, 125.1, 123.8, 121.5, 112.7, 110.2, 68.4, 64.5, 61.8, 27.3, 20.7 14.8, 14.3 ppm. GC/MS (EI): m/z (%): 277 (22) [M⁺], 195 (100), 179 (1), 167 (24), 140 (13), 125 (5), 55 (38).

Odor description (1% solution in EtOH on paper blotter, 24 h): vanilla (sweet, gourmand, carnation floral, chestnut cream)

### Example 48: (Z)-2-ethoxy-4-(ethoxymethyl)-1-(hex-3-en-1-yloxy)benzene

### a) (Z)-(3-ethoxy-4-(hex-3-en-1-yloxy)phenyl)methanol

To a solution of (Z)-3-ethoxy-4-(hex-3-en-1-yloxy)benzaldehyde (24.0 g, 96.6 mmol, 1.0 equiv) in methanol (200 ml) was added sodium borohydride (1.8 g, 48.3 mmol, 0.5 equiv) portionwsie at 10° C. and stirred for 16 hours under argon atmosphere at rt. The solvent was removed by rotary evaporation, and water 200 mL was added, the solution was extracted with MTBE (150 mL*3), the organic layer was dried with MgSO₄, filtered, and concentrated, the residue was purified by silica gel column chromatography (PE:MTBE=4:1) to give (Z)-(3-ethoxy-4-(hex-3-en-1-yloxy)phenyl)methanol (85.0 mmol, 22.0 g, yield: 88%) as colorless liquid. ¹H NMR (400 MHz, CDCl₃) δ 6.90 (s, 1H), 6.84 (s, 2H), 5.54-5.41 (m, 2H), 4.57 (d, J=5.5 Hz, 2H), 4.07 (q, J=7.0 Hz, 2H), 3.98 (t, J=7.2 Hz, 2H), 2.60-2.55 (m, 2H), 2.14-2.07 (m, 2H), 1.98 (t, J=5.7 Hz, 1H), 1.43 (t, J=7.0 Hz, 3H), 0.98 (t, J=7.5 Hz, 3H) ppm. ¹³C NMR (101 MHz, CDCl₃) δ 149.0, 148.3, 134.4, 133.9, 124.0, 119.6, 113.7, 112.8, 68.8, 65.2, 64.6, 27.5, 20.7, 14.9, 14.3 ppm. GC/M S (EI): m/z (%): 250 (24) [M+], 194 (1), 168 (100), 153 (2), 140 (27), 122 (36), 107 (4), 93 (17), 77 (6), 65 (12), 55 (43).

### b) (Z)-2-ethoxy-4-(ethoxymethyl)-1-(hex-3-en-1-yloxy)benzene

To a solution of sodium hydride (0.7 g, 56% Wt, 16.0 mmol, 2.0 equiv) in DMF (50 mL) was added (Z)-(3-ethoxy-4-(hex-3-en-1-yloxy)phenyl)methanol (2.0 g, 8.0 mmol, 1.0 equiv) at 0° C. and the mixture was stirred for 5 hours under argon atmosphere at r.t. Then bromoethane (2.6 g, 24.0 mmol, 3.0 equiv) was added to the reaction solution at 0° C. and the resulting mixture was stirred at r.t for 16 h. The reaction was quenched by adding water, then extracted with MTBE (100 mL*3), the organic layer was dried with MgSO4, filtered, and concentrated, the residue was purified by silica gel column chromatography (PE:MTBE=98:2) and give (Z)-2-ethoxy-4-(ethoxymethyl)-1-(hex-3-en-1-yloxy)benzene (1.2 g, 4.2 mmol, yield: 52%) as colorless liquid.

¹H NMR (400 MHz, CDCl₃) δ 6.90 (s, 1H), 6.84 (s, 2H), 5.54-5.41 (m, 2H), 4.42 (s, 2H), 4.09 (q, J=7.0 Hz, 2H), 3.98 (t, J=7.2 Hz, 2H), 3.51 (q, J=7.0 Hz, 2H), 260-2.55 (m, 2H), 2.14-2.06 (m, 2H), 1.44 (t, J=7.0 Hz, 3H), 1.23 (t, J=7.0 Hz, 3H), 0.98 (t, J=7.5 Hz, 3H). ¹³C NMR (101 MHz, CDCl₃) δ 148.9, 148.3, 134.4, 131.4, 124.1, 120.4, 113.6, 113.4, 72.6, 68.8, 65.5, 64.5, 27.5, 20.7, 15.3, 14.9, 14.3 ppm. GC/MS (EI): m/z (%): 278 (46) [M⁺], 196 (100), 179 (5), 151 (89), 135 (16), 123 (57), 83 (17), 55 (79).

Odor description (1% solution in EtOH on paper blotter, 24 h): spicy (eugenol, smokey), powdery (vanilla)

### Example 49: (Z)-2-ethoxy-1-(hex-3-en-1-yloxy)-4-(methoxymethyl)benzene

The compound was obtained from (Z)-(3-ethoxy-4-(hex-3-en-1-yloxy)phenyl)methanol (3.0 g, 12.0 mmol, 1.0 equiv) and iodomethane (5.1 g, 36.0 mmol, 3.0 equiv) according to the process of example 48 as colorless liquid (5.5 mmol, 1.5 g, 46% yield).

¹H NMR (400 MHz, CDCl₃) δ 6.89 (s, 1H), 6.86-6.82 (m, 2H), 5.59-5.47 (m, 1H), 5.47-5.37 (m, 1H), 4.37 (s, 2H), 4.09 (q, J=7.0 Hz, 2H), 3.99 (t, J=7.2 Hz, 2H), 3.36 (s, 3H), 2.60-2.55 (m, 2H), 2.14-2.07 (m, 2H), 1.44 (t, J=7.0 Hz, 3H), 0.98 (t, J=7.5 Hz, 3H) ppm. ¹³C NMR (101 MHz, CDCl₃) δ 148.9, 148.4, 134.4, 131.0, 124.0, 120.5, 113.5, 113.4, 74.6, 68.8, 64.5, 57.8, 27.5, 20.7, 14.9, 14.3 ppm. GC/MS (EI): m/z (%): 264 (31) [M⁺], 182 (100), 151 (61), 137 (14), 123 (3565 (11), 55 (43).

Odor description (1% solution in EtOH on paper blotter, 24 h): spicy (eugenol, smokey), powdery (vanilla)

### Example 50: (Z)-2-ethoxy-1-(hex-3-en-1-yloxy)-4-(isopropoxymethyl)benzene

The compound was obtained from (Z)-(3-ethoxy-4-(hex-3-en-1-yloxy)phenyl)methanol (3.0 g, 12.0 mmol, 1.0 equiv) and 2-bromopropane (4.4 g, 36.0 mmol, 3.0 equiv) according to the process of example 48 as colorless liquid (0.5 mmol, 0.2 g, 4% yield).

¹H NMR (400 MHz, CDCl₃) δ 6.90 (s, 1H), 6.84 (s, 2H), 5.57-5.47 (m, 1H), 5.47-5.36 (m, 1H), 4.42 (s, 2H), 4.09 (q, J=7.0 Hz, 2H), 3.98 (t, J=7.2 Hz, 2H), 3.66 (dt, J=12.2, 6.1 Hz, 1H), 2.60-2.54 (m, 2H), 2.14-2.06 (m, 2H), 1.44 (t, J=7.0 Hz, 3H), 1.20 (d, J=6.1 Hz, 6H), 0.98 (t, J=7.5 Hz, 3H) ppm. ¹³C NMR (101 MHz, CDCl₃) δ 148.9, 148.2, 134.3, 132.0, 124.1, 120.2, 113.7, 113.4, 70.6, 69.9, 68.8, 64.5, 27.5, 22.2, 20.7, 14.9, 14.3 ppm. GC/MS (EI): m/z (%): 292 (73) [M⁺], 233 (3), 210 (60), 152 (100), 139 (25), 123 (56), 55 (54).

Odor description (1% solution in EtOH on paper blotter, 24 h): spicy (eugenol, smokey), powdery (vanilla), green

### Example 51: 4-(cyclohex-3-en-1-yloxy)-3-ethoxybenzaldehyde

The compound was obtained from 3-ethoxy-4-hydroxybenzaldehyde (10.0 g, 60.2 mmol, 1.0 equiv) and cyclohexane-1,4-diol (9.1 g, 78.3 mmol, 1.3 equiv) according to the process of example 19 as colorless liquid (8.1 mmol, 2.0 g, 13% yield).

¹H NMR (400 MHz, CDCl₃) δ 9.83 (s, 1H), 7.49-7.33 (m, 2H), 7.02 (d, J=8.7 Hz, 1H), 5.88-5.49 (m, 2H), 4.74-4.53 (m, 1H), 4.12 (q, J=7.0 Hz, 2H), 2.59-2.44 (m, 1H), 2.39-2.23 (m, 2H), 2.22-2.03 (m, 2H), 1.95-1.79 (m, 1H), 1.44 (t, J=7.0 Hz, 3H) ppm. ¹³C NMR (101 MHz, CDCl₃) δ 190.9, 153.4, 150.1, 130.2, 126.9, 126.2, 123.6, 114.6, 111.9, 74.4, 64.7, 31.0, 27.6, 23.7, 14.7 ppm. GC/MS (EI): m/z (%): 246 (24) [M⁺], 192 (1), 166 (100), 149 (11), 138 (78), 81 (95), 53 (25).

Odor description (1% solution in EtOH on paper blotter, 24 h): powdery (vanilla)

### Example 52: 4,4′-(hex-3-ene-1,6-diylbis(oxy))bis(3-ethoxybenzaldehyde)

The compound was obtained from 3-ethoxy-4-hydroxybenzaldehyde (10.0 g, 60.2 mmol, 2.5 equiv) and 1,6-dibromohex-3-ene (5.8 g, 24.1 mmol, 1.0 equiv) according to the process of example 1 as colorless liquid (2.7 mmol, 1.1 g, 22% yield, E/Z mixture, E:Z=2:1).

¹H NMR (400 MHz, CDCl₃, E/Z mixture) δ 9.83 (s, 2H), 7.52-7.31 (m, 4H), 6.97-6.92 (m, 2H), 6.26-5.55 (m, 2H), 4.77-3.98 (m, 8H), 2.79-2.55 (m, 2H), 2.45-2.30 (m, 1H), 2.03-1.96 (m, 1H), 1.60-1.38 (m, 6H) ppm. ¹³C NMR (101 MHz, CDCl₃, E isomer) δ 191.0, 154.2, 149.2, 130.0, 128.4, 126.6, 111.9, 110.9, 64.5, 64.4, 32.4, 14.7 ppm.

GC/M S (EI): m/z (%): 412 (6) [M⁺], 246 (11), 177 (23), 137 (40), 109 (16), 81 (100), 67 (15), 53 (15).

Odor description (1% solution in EtOH on paper blotter, 24 h): powdery (vanilla)

### Example 53: 3-ethoxy-4-(pent-4-en-1-yloxy)benzaldehyde—Comparative Example

The compound was obtained from 3-ethoxy-4-hydroxybenzaldehyde (8.0 g, 48.1 mmol, 1.0 equiv) and 5-bromopent-1-ene (9.3 g, 62.6 mmol, 1.3 equiv) according to the procedure of example 1 as colorless liquid (43.9 mmol, 10.6 g, 91% yield).

¹H NMR (400 MHz, CDCl₃): δ 9.83 (s, 1H), 7.38-7.44 (m, 2H), 6.96 (d, J=8.1 Hz, 1H), 5.80-5.87 (m, 1H), 5.00-5.11 (m, 2H), 4.07-4.18 (m, 4H), 2.30-2.24 (m, 2H), 2.01-1.94 (m, 2H), 1.47 (t, J=7.0 Hz, 3H) ppm. ¹³C NMR (101 MHz, CDCl₃) δ 191.0, 154.4, 149.2, 137.6, 130.0, 126.6, 115.4, 111.9, 110.9, 68.4, 64.6, 30.0, 28.1, 14.7 ppm. GC/M S (EI): m/z (%): 234 (65) [M⁺], 206 (1), 166 (65), 149 (10), 138 (100), 81 (25), 69 (18).

### Example 54: (E)-3-ethoxy-4-(hex-2-en-1-yloxy)benzaldehyde—comparative example

The compound was obtained from 3-ethoxy-4-hydroxybenzaldehyde (4.0 g, 24.1 mmol, 1.0 equiv) and (E)-1-bromohex-2-ene (5.9 g, 36.1 mmol, 1.5 equiv) according to the procedure of example 1 as colorless liquid (18.1 mmol, 4.5 g, 75% yield).

1H NMR (400 MHz, CDCl₃): δ 9.83 (s, 1H), 7.41-7.40 (m, 2H), 6.98 (d, J=8.1 Hz, 1H), 5.81-5.90 (m, 1H), 5.68-5.76 (m, 1H), 4.64 (d, J=5.8 Hz, 2H), 4.15 (q, J=7.0 Hz, 2H), 1.98-2.16 (m, 2H), 1.38-1.51 (m, 5H), 0.85-0.97 (m, 3H) ppm. ¹³C NMR (101 MHz, CDCl₃) δ 190.9, 154.0, 149.2, 136.1, 130.0, 126.4, 124.1, 112.3, 110.6, 69.8, 65.2, 34.4, 22.5, 14.6, 13.6 ppm. GC/M S (EI): m/z (%): 248 (15) [M+], 219 (1), 166 (100), 138 (70), 81 (25), 55 (35).

### Example 55: Application in Liquid Detergent

### a) Sample Preparation

0.2% by weight of ethyl vanillin (3-ethoxy-4-hydroxybenzaldehyde, sample A) or (Z)-3-ethoxy-4-(hex-3-en-1-yloxy)benzaldehyde (compound of example 1, sample B) were incorporated respectively into unperfumed liquid detergent base by magnetic stirring at room temperature for 24 h.

### b) Color Stability

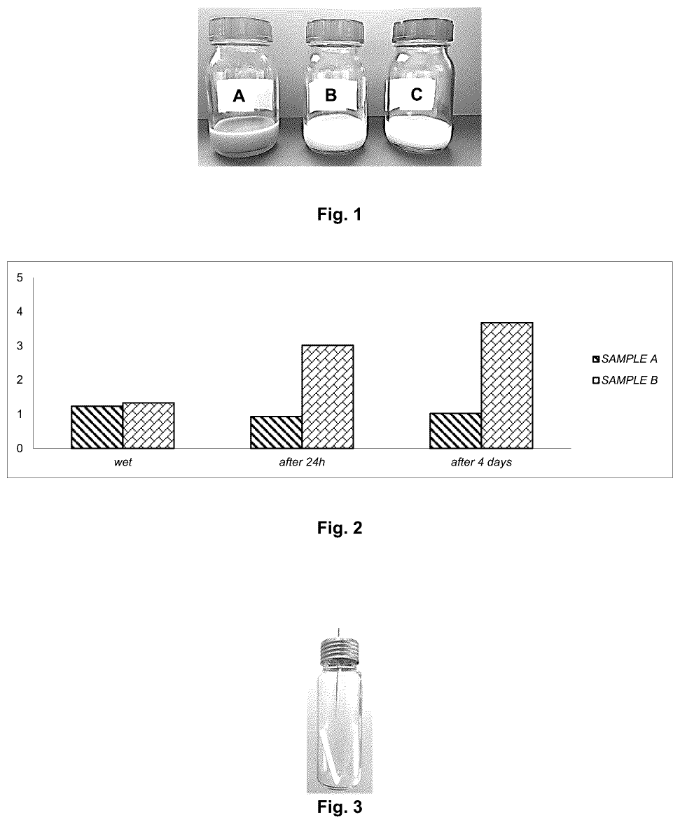

The above prepared samples were visually inspected in comparison to an unperfumed base sample (sample C). The ethylvanillin sample (A) showed strong brown-purple coloration, whereas the sample containing the precursor (B) was optically identical to the unperfumed base (C). All three samples are shown in FIG. 1.

### c) Washtest and Sensory Evaluation

A 40° C. machine wash cycle was performed using 55 g of the above prepared liquid detergent samples A and B and odor-neutral cotton/elastan mixed fabric T-shirts. The wet and line-dried fabric (1 and 4 days) was assessed by a panel of 4-6 experts with regard to odor intensity and quality. The odor intensity was recorded on an intensity scale of 0 (odorless) to 5 (extremely strong). As can be seen from Table 1 below, virtually no odor was released from the T-shirts washed with the colored ethyl vanillin detergent base, whereas the T-shirts washed with the color-neutral detergent base containing (Z)-3-ethoxy-4-(hex-3-en-1-yloxy)benzaldehyde exhibited a strong vanilla, sweet, powdery and creamy fragrance after 1 and 4 days. The difference in odor strength between the two samples after 1 and 4 days was highly significant (Student's t-test, p<0.01).

TABLE 1

| Sample | wet | 1 day | 4 days |
|---|---|---|---|
| Ethylvanillin (A) | 1.2 | 0.9 | 1 |
| (Z)-3-ethoxy-4-(hex-3-en-1- | 1.3 | 3 | 3.7 |
| yloxy)benzaldehyde (B) | | | |
| significance of difference | | p = 0.001 | p = 0.003 |

The results are also visualized in FIG. 2. The experiment demonstrates that free ethylvanillin is not suitable for application in liquid detergent, as it colors the base and, due to its high water solubility, does not impart any fragrance to washed and dried fabric. These technical limitations are overcome by the compound of the present invention, which is not colorizing and imparts a strong vanilla fragrance on dry fabric.

### Example 56: Application in Liquid Detergent (Position of Double Bond in Side Chain)

In order to highlight the importance of the position of the double bond, a comparative assessment in liquid detergent application (as described in Example 55a and c) was carried out with ethylvanillin ethers (compounds of formula IV) having unsaturated side chains with the double bond placed in positions A-2 or A-4 in comparison to the compounds of the current invention which have the double bond positioned at A-3. The odour intensity was recorded on an intensity scale of 0 (odorless) to 5 (extremely strong). The results are summarized in Table 2.

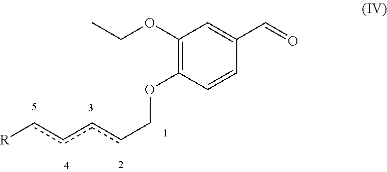

TABLE 2

| | | length | position | | | |
|---|---|---|---|---|---|---|
| | | of side | of double | | 1 | 4 |
| Entry | Compound | chain | bond | wet | day | days |
| 1 | 3-ethoxy-4- | C₅ | Δ-4 | 1.5 | 1.3 | 1.2 |
| | (pent-4-en-1-yloxy) | | | | | |
| | benzaldehyde | | | | | |
| | (Example 53) | | | | | |
| 2 | 3-ethoxy-4- | | Δ-3 | 1.1 | 3.2 | 3.8 |
| | (pent-3-en-1-yloxy) | | | | | |
| | benzaldehyde | | | | | |
| | (Example 33) | | | | | |
| 3 | 3-ethoxy-4- | C₆ | Δ-2 | 1.2 | 1.8 | 1.4 |
| | (hex-2-en-1-yloxy) | | | | | |
| | benzaldehyde | | | | | |
| | (Example 54) | | | | | |
| 4 | 3-ethoxy-4- | | Δ-3 | 3.2 | 3.2 | 3.6 |
| | (hex-3-en-1-yloxy) | | | | | |
| | benzaldehyde | | | | | |
| | (Example 1) | | | | | |

The results show that only compounds of the present invention with the double bond in the Δ-3 position of the side chain (homoallylic ethers, entries 2 and 4) release a strong odor on dry fabric. In contrast, only very weak odors were detected on dry fabric washed with similar compounds with identical chain lengths having the double bond either in the Δ-4 or Δ-2 position (entries 1 and 3).

### Example 57: Application in Liquid Detergent—Generation of Different Odor Profiles

Liquid detergent samples were prepared and used in washtests and subsequent sensory evaluation of the washed and dried fabric as described in Example 45a and c. The odor intensity was recorded on an intensity scale of 0 (odorless) to 5 (extremely strong). The results are summarized in Table 3.

TABLE 3

| Entry | | wet | 1 day | 4 days | odor on dry fabric |
|---|---|---|---|---|---|
| 1 | 3-ethoxy-4-((4-methylpent-3-en-1-yl)oxy) | 1.5 | 3.3 | 3.4 | vanilla |
| | benzaldehyde (Example 34) | | | | |
| 2 | methyl (Z)-4-(hex-3-en-1-yloxy)-2-hydroxy- | 1.0 | 1.7 | 3.3 | oakmoss |
| | 3,6-dimethylbenzoate (Example 9) | | | | |
| 3 | 4-(4-(hex-3-en-1-yloxy)phenyl)butan-2-one | 3.0 | 2.8 | 3.6 | raspberry |
| | (Example 6) | | | | |
| 4 | 1-(((Z)-hex-3-en-1-yl)oxy)-2-methoxy-4- | 2.0 | 1.9 | 3.0 | spicy, vanilla |
| | ((E)-prop-1-en-1-yl)benzene (Example 11) | | | | |
| 5 | 4-(cyclopent-3-en-1-yloxy)-3- | n.d. | 3.0 | 3.7 | vanilla, powdery |
| | ethoxybenzaldehyde (Example 20) | | | | |
| 6 | (Z)-4-allyl-1-(hex-3-en-1-yloxy)-2- | 1.6 | 2.4 | 2.5 | spicy, clove, |
| | methoxybenzene (Example 10) | | | | gingerbread |

The results illustrate that strong fragrance intensities are achieved on dry fabric with compounds of the current invention. The odor direction is dominated by the given phenolic fragrant compound HX. In some cases, it can be slightly modulated by the side chain or its fragments.

### Example 58: Headspace Analysis of Released Volatiles

A solution of the compound of formula (I) (100 g, 1.0 mg/mL solution in MTBE) was evenly applied through a displacement pipette to a paper strip (1 cm×8 cm). The paper strip was left to dry (1 h at room temperature), then folded in the middle and inserted in “V”-shape into a 20 mL headspace vial (FIG. 3). The vial was closed with a septum screwcap and placed in a cabinet equipped with a fluorescent daylight imitation lamp and mirrored walls for 24 h (temperature 27-28° C.).

Extraction and analysis of the released organic volatiles was effected in automated mode on a Trace 1310 gas chromatograph equipped with a TriPlus RSH autosampler (Thermo Fisher Scientific), coupled to a ISQ LT mass spectrometer (Thermo Fisher Scientific). The extraction of the organic volatiles from the headspace with SPME (solid phase microextraction) was carried out during 1 h at 40° C. using a 50/30 μm divinylbenzene/carboxen/polydimethylsiloxane SPME fibre (DVB/CAR/PDMS, Supelco P/N 57298-U), and subsequently desorbed in splitless mode at 250° C. for 1 min. GC-MS analysis (gas chromatography with mass spectrometric detection) was carried out with a VF-WAXms capillary column (Agilent, 30 m length, 0.25 mm I.D., 0.25 μm film thickness). Helium was used as a carrier gas with a constant flow rate of 1 ml/min. The GC oven temperature was programmed from 35° C. with 2-min hold to 250° C. at 5° C./min with a 10-min final temperature hold. The mass spectrometer was operated at 70 eV in EI mode over a m/z range of 33-350, scanned at 0.2 s intervals and with a temperature of transfer line and ion source of 230° C. and 220° C., respectively. The main sensorially active volatiles released from the compounds of formula (I) are reported in Table 4 (indicated are relative peak area %; in bold: main odor vector).

TABLE 4

| Precursor | Released Volatiles |
|---|---|
| (Z)-3-ethoxy-4-(hex-3-en-1- | (Z)-hex-3-en-1-ol (5.6%), ethyl vanillin |
| yloxy)benzaldehyde (Example 1) | (0.3%) |
| 3-ethoxy-4-((4-methylpent-3-en-1- | 3-methylbut-2-enal (0.1%), 4-methylpent-3- |
| yl)oxy)benzaldehyde (Example 34) | en-1-ol (2.7%), 5,5-dimethylfuran-2(5H)-one |
| | (0.3%), ethyl vanillin (0.5%) |
| 3-ethoxy-4-(pent-3-en-1- | (E,Z)-pent-3-en-1-ol (1.2%), ethyl vanillin |
| yloxy)benzaldehyde (Example 33) | (0.7%) |
| (Z)-4-allyl-1-(hex-3-en-1-yloxy)-2- | (Z)-hex-3-en-1-ol (1.9%), eugenol (1.0%) |
| methoxybenzene (Example 10) | |
| (Z)-2-ethoxy-1-(hex-3-en-1-yloxy)-4- | (Z)-hex-3-en-1-ol (0.8%), 2-ethoxy- |
| (isopropoxymethyl)benzene | 4(isopropoxymethyl)phenol (« Propyl |
| (Example 50) | Diantilis », 17.1%) |

It should be noted that the indicated relative peak area % values are not related to molar release rates. The relative amount of each volatile in the gas phase depends mainly on volatility. Therefore, the sensory active phenols appear as minor components. The sensorially active phenols were observed also in samples which were not exposed to a light source under otherwise identical conditions, indicating that the release can occur upon exposure to ambient air by oxidation in the presence or absence of light.

### Example 59: Assessment of Biodegradability

The results of the biodegradability assessment by the manometric respirometry test (OECD guideline for the testing of materials No. 301F, Paris 1992) are summarized in Table 5.

TABLE 5

| Compound | | Biodegradation | |
|---|---|---|---|
| of example | Compound name | in % | result |
| 1 | (Z)-3-ethoxy-4-(hex-3-en-1- | 61 | inherently |
| | yloxy)benzaldehyde | | biodegradable |
| 33 | 3-ethoxy-4-(pent-3-en-1- | 66 | inherently |
| | yloxy)benzaldehyde | | biodegradable |
| 34 | 3-ethoxy-4-((4-methylpent- | 69 | inherently |
| | 3-en-1-yl)oxy)benzaldehyde | | biodegradable |
| 8 | (Z)-4-(hex-3-en-1-yloxy)-3- | 92 | readily |
| | methoxybenzaldehyde | | biodegradable |
| 6 | 4-(4-(hex-3-en-1- | 82 | readily |
| | yloxy)phenyl)butan-2-one | | biodegradable |
| 18 | 4-(4-((4-methylpent-3-en-1- | 91 | readily |
| | yl)oxy)phenyl)butan-2-one | | biodegradable |
| 20 | 4-(cyclopent-3-en-1-yloxy)- | 74 | inherently |
| | 3-ethoxybenzaldehyde | | biodegradable |

The results show that compounds of the present invention with varying phenol moieties and side chains are biodegradable. A compound can be classified biodegradable, if it reaches the pass level of 60% oxygen consumption of theory required for complete mineralization.

A compound is readily biodegradable, if the pass level is reached within 10 days within the 28-day period of the test. The 10-day window begins when the degree of biodegradation has reached 10%. If the pass level is obtained after 28-day period of the test, the compound can be classified as inherently biodegradable.

### Example 60: methyl 5-(2-ethoxy-4-formylphenoxy)pent-2-enoate

To a solution of (Z)-3-ethoxy-4-(hex-3-en-1-yloxy)benzaldehyde (10.0 g, 40 mmol) and methyl but-2-enoate (8.1 g, 80 mmol) in DCE (50 mL) was added Zhan catalyst-1b (0.3 g, 0.4 mmol) and stirred at 70° C. for 16 hours under argon. The solvent was removed by rotary evaporation, and the residue was purified by column chromatography on silica gel (PE:MTBE=7:3) to give 5-(2-ethoxy-4-formylphenoxy)pent-2-enoate (9.0 mmol, 3.7 g, yield: 22%) as white solid.

¹H NMR (400 MHz, CDCl₃, mixture of E/Z isomers) δ 9.92-9.77 (m, 1H), 7.47-7.33 (m, 2H), 7.11-6.88 (m, 2H), 6.08-5.86 (m, 1H), 4.28-4.03 (m, 4H), 3.81-3.67 (m, 3H), 2.84-2.72 (m, 2H), 1.47 (t, J=7.0 Hz, 3H) ppm. ¹³C NMR (101 MHz, CDCl₃, mixture of E/Z isomers) δ 191.0, 191.0, 166.7, 154.1, 153.8, 149.4, 145.3, 144.2, 130.5, 130.3, 126.5, 126.4, 123.5, 121.5, 112.3, 112.2, 111.1, 111.0, 67.8, 67.1, 64.6, 64.6, 51.6, 51.2, 31.8, 28.8, 14.7, 14.6 ppm.

Odour description (1% solution in EtOH on paper blotter, 24 h): sweet powdery gourmand, vanilla, coffee, creamy, vanilla, milky

### Example 61: ethyl 2-(2-ethoxy-4-formylphenoxy)pent-4-enoate

### a) Ethyl 2-hydroxypent-4-enoate

To a solution of allyltrimethylsilane (11.2 g, 98 mmol) and ethyl 2-oxoacetate (5.0 g, 49 mmol) in DCM (100 mL) was added boron trifluoride diethyl etherate (10.4 g, 74 mmol) in DCM (50 mL) slowly at room temperature and stirred for 16 hours. The mixture was quenched by adding aqueous NH₄Cl solution, and extracted with MTBE (3*100 mL), the organic layers were combined, dried over MgSO₄, filtered, and solvent was removed by rotary evaporation. The residue was purified by column chromatography on silica gel (PE:MTBE=9:1) to give ethyl 2-hydroxypent-4-enoate (2.3 g, yield: 33%) as light yellow liquid.

¹H NMR (400 MHz, CDCl₃) δ 5.89-5.74 (m, 1H), 5.20-5.09 (m, 2H), 4.35-4.16 (m, 3H), 2.89 (d, J=5.9 Hz, 1H), 2.64-2.35 (m, 2H), 1.33-1.25 (m, 3H) ppm. ¹³C NMR (101 MHz, CDCl₃) δ 174.4, 132.5, 118.7, 70.0, 61.7, 38.7, 14.2 ppm.

### b) Ethyl 2-(2-ethoxy-4-formylphenoxy)pent-4-enoate

Ethyl 2-(2-ethoxy-4-formylphenoxy)pent-4-enoate was obtained from 3-ethoxy-4-hydroxybenzaldehyde (3.2 g, 19 mmol) and ethyl 2-hydroxypent-4-enoate (2.5 g, 17 mmol) according to the procedure of example 19 as colorless liquid (8.4 mmol, 2.5 g, 48% yield).

¹H NMR (400 MHz, CDCl₃) δ 9.82 (s, 1H), 7.44-7.30 (m, 2H), 6.94 (d, J=8.1 Hz, 1H), 6.07-5.84 (m, 1H), 5.32-5.04 (m, 2H), 4.80 (t, J=6.1 Hz, 1H), 4.29-4.03 (m, 4H), 2.76 (t, J=6.5 Hz, 2H), 1.44 (t, J=7.0 Hz, 3H), 1.24 (t, J=7.1 Hz, 3H) ppm. ¹³C NMR (101 MHz, CDCl₃) δ 190.8, 170.3, 152.9, 149.9, 132.2, 131.4, 125.7, 118.7, 115.7, 112.0, 77.6, 64.7, 61.3, 37.1, 14.6, 14.2 ppm. GC/MS (EI): m/z (%): 292 (100) [M⁺], 264 (1), 219 (9), 166 (38), 149 (42), 137 (67), 99 (61), 81 (29).

Odour description (1% solution in EtOH on paper blotter, 24 h): sweet powdery gourmand (vanilla, coffee) creamy (vanilla, milky)

### Example 62: methyl 2-hydroxy-3,6-dimethyl-4-((4-methylpent-3-en-1-yl)oxy)benzoate

The compound was obtained from methyl 2,4-dihydroxy-3,6-dimethylbenzoate (1.5 g, 7.7 mmol) and 5-bromo-2-methylpent-2-ene (1.4 g, 8.4 mmol) according to the process of example 1 as white solid (0.5 g, 20% yield).

1H NMR (400 MHz, CDCl3) β 11.75 (s, 1H), 6.17 (s, 1H), 5.14 (t, J=7.2 Hz, 1H), 3.92-3.81 (m, 5H), 2.50-2.31 (m, 5H), 2.01 (s, 3H), 1.69-1.55 (m, 6H) ppm. 13C NMR (101 MHz, CDCl3) β 171.6, 161.2, 159.9, 138.9, 133.5, 118.5, 110.0, 105.8, 104.2, 66.8, 50.7, 27.3, 24.7, 23.6, 16.8, 6.8 ppm. GC/MS (EI): m/z (%): 278 (2) [M⁺], 247 (1), 231 (4), 196 (22), 164 (47), 83 (66), 55 (100).

Odour description (1% solution in EtOH on paper blotter, 24 h): mossy (evernyl)

### Example 63: methyl 2-hydroxy-3,6-dimethyl-4-(pent-3-en-1-yloxy)benzoate

The compound was obtained from methyl 2,4-dihydroxy-3,6-dimethylbenzoate (4.0 g, 20 mmol) and 5-bromopent-2-ene (3.0 g, 20.4 mmol) according to the procedure of example 1 as white solid (3.1 g, 58% yield).

1H NMR (400 MHz, CDCl3) β 11.75 (s, 1H), 6.25-6.06 (m, 1H), 5.60-5.32 (m, 2H), 3.96-3.79 (m, 5H), 2.54-2.30 (m, 5H), 1.99 (s, 3H), 1.67-1.54 (m, 3H) ppm. ¹³C NMR (101 MHz, CDCl₃) δ 172.6, 162.2, 160.9, 140.0, 127.9, 126.6, 111.1, 106.8, 105.3, 67.9, 51.7, 32.6, 24.6, 18.0, 7.8 ppm. GC/MS (EI): m/z (%): 264 (27) [M⁺], 217 (29), 196 (76), 164 (100), 136 (47), 69 (51).

Odour description (1% solution in EtOH on paper blotter, 24 h): mossy, evernyl, dry powdery

### Example 64: Application in Shampoo Base

A hair shampoo sample (20 g) was prepared by adding 0.2% by weight of 3-ethoxy-4-(hex-3-en-1-yloxy)benzaldehyde (compound of example 3) to unperfumed clear hair shampoo base and mixing on a bottle roller for 24 h at room temperature. An odour-neutral human hair swatch was wetted with warm tap water and lathered delicately with 2 mL of the above hair shampoo sample for 30 sec by hand wearing gloves. The lathered hair swatch was left in a plastic bowl for 2 min, then rinsed under running tap water for 20 sec. After removing excess water by squeezing the hair swatch between two fingers, it was left to dry in open air. The odour of the wet and dried hair swatch (1 and 3 days) was assessed by a panel of 7 experts. Five out of seven experts perceived a vanilla note on dry hair after 1 and 3 days.

The experiment demonstrates that precursors of the current invention are capable of releasing fragrance on dry hair.

### Example 65: Malodour Reduction

Fabrics (18 cm×14 cm cotton jersey) were stripped before use. They were then washed in European washing machines (40° C.—1,000 spin/min) with one unit dose (65 g) used per wash load. The unit dose contained 2% of a 10% solution of the compound of formula 1 in trietylcitrate.
a) Compound of example 33
b) Compound of example 34
c) Compound of example 1

The total wash load was 1.6 kg made up of test cloths and hand towels (100% cotton) as ballast. The cloth was line dried and left overnight in a perfume free room at 25° C., left to hang for 3 days and then wrapped in foil the day prior to the test (day 3). 30 minutes before the assessment, 50 μL of the pure Male Sweat malodour was placed on top of a cotton pad and introduced into a 500 ml powder jar. One cotton jersey square was then placed over the top of the powder jar and secured with a rubber band. Every sample tested was coded with a random code number.

The malodour and fragrance perceived intensities were assessed by the trained sensory panel using a 0-100 line scale. All malodour and fragrance perceived intensities were scaled against the malodour control sample. The malodour perceived intensity was set at 40 for Male Sweat malodour and the fragrance perceived intensity was set at 0.

The order of samples assessed by the panelists was pre-determined using a balanced randomisation. The samples were assessed in a sequential monadic way. The products were assessed twice by 14 panelists, thus giving 28 assessments per sample.

Before analysis, the data was checked for outliers and extreme violations to normality.

Reliability of the data was checked and data of panelists with poor discrimination, consensus or repeatability may have been removed from the analysis. The data was analysed using analysis of variance (ANOVA). A 5% significance level (95% confidence level) was used. Separate ANOVAs were carried out for malodour and fragrance intensity. The ANOVA have product, panelist, and replicate as factors. Pairwise comparisons of the products were done using the Benjamini-Hochberg correction for multiple testing. The estimated product means are reported. It is indicated with letters if the product means are statistically significantly different from each other: if they are not, then they share the same letter.

### a) Perceived Malodour Intensity

| | | Perceived | |
|---|---|---|---|
| | | Malodour | Significance of |
| Sample | Composition | Intensity | Differences |
| 1 | Compound of example 34 + | 20.6 | A |
| | Male Sweat Malodour | | |
| 2 | Compound of example 33 + | 26.5 | B |
| | Male Sweat Malodour | | |
| 3 | Compound of example 1 + | 29.5 | B |
| | Male Sweat Malodour | | |
| 4 | Male Sweat Malodour | 37.0 | C |

Where the same letter is shown in the “significance of differences” column there are no statistically significant differences between the relevant figures.

When assessed from 4 day dry cloth, fabrics washed with any of the three products significantly reduced the perception of the Male Sweat Malodour (in comparison with sample 4). Sample 2 was not significantly different in perceived malodour intensity (PMI) than sample 3, but these two samples had a significantly higher PMI than sample 1.

### b) Perceived Fragrance Intensity

| | | Perceived | |
|---|---|---|---|
| | | Fragrance | Significance of |
| Sample | Sample | Intensity | Differences |
| 5 | Male Sweat Malodour | 3.0 | A |
| 6 | Compound of example 1 + | 18.3 | B |
| | Male Sweat Malodour | | |
| 7 | Compound of example 33 + | 22.3 | B |
| | Male Sweat Malodour | | |
| 8 | Compound of example 34 + | 31.1 | C |
| | Male Sweat Malodour | | |

Where the same letter is shown in the “significance of differences” column there are no statistically significant differences between the relevant figures.

When the 4 day dry cloth was assessed against sample 5 in the presence of the Male Sweat Malodour, the fabric washed with sample 8 was significantly stronger in perceived fragrance intensity (PFI) than fabric washed with sample 6 or sample 7, which were not perceived to be significantly different in PFI.

The compounds of the present invention significantly reduced the perceived intensity of the sweat malodour and were perceivable in the presence of the malodour.

1. A method of generating a phenolic fragment compound HX which is a compound of formula (II) 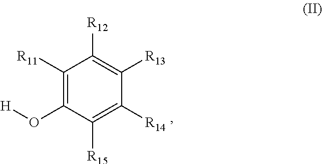
the method comprising exposing a compound of formula (I) to ambient air in the presence or absence of light 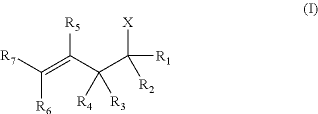
wherein
R₁-R₇ is independently selected from the group consisting of H, linear C1-C4 hydrocarbon groups and branched C1-C4 hydrocarbon groups, wherein the linear and branched C1-C4 hydrocarbon groups independently bear up to 2 heteroatoms selected from the group consisting of O, S and N, and bear up to one double bond or triple bond, and bearing up to one ring; or
R₁ and R₆, R₁ and R₃, R₁ and R₅, R₃ and R₅, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, while the other substituents have the same meaning as previously defined;
and the sum of all carbon atoms of R₁-R₇ is limited to 4;
or R₇ is —CR′₃R′₄—CR′₁R′₂—X′, wherein R′₁, R′₂, R′₃, R′₄, and X′ are selected from the corresponding groups of R₁, R₂, R₃, R₄, and X, while the other substituents have the same meaning as previously defined, and the sum of all carbon atoms of R₁-R₆ and R′₁—R′₄ is limited to 4;
and wherein X is representing the following fragment 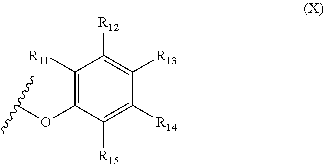
wherein
R₁₁-R₁₅ is independently selected from the group consisting of H, linear or branched C1-C5 alkyl, linear or branched C2-C5 alkenyl, linear or branched C2-C5 alkynyl, OH, C1-C4-alkoxy, CHO, C(O)Me, C(O)Et, hexanoyl, heptanoyl, octanoyl, propanoyl, 3-phenylpropanoyl, 5-methylhexanoyl, (CH₂)₂C(O)CH₃, (4-methyl-3,6-dihydro-2H-pyran-2-yl), CH₂—O-Me, CH₂—O-Et, CH₂—O-iPr, CH₂—OH, Cl, Br, phenyl, and CH(OR₁₆)(OR₁₇) wherein R₁₆ and R₁₇ are independently selected from the group consisting of C1-C5 alkyl, R₁₆ and R₁₇ form together with the carbon atoms of the chain they are attached to a 5 or 6 membered ring, C═N—OR₁₈ (R₁₈═C1-C5 alkyl), and CO₂R₁₉, wherein R₁₉ is selected from the group consisting of linear or branched C1-C8 alkyl, linear or branched C2-C8 alkenyl, linear or branched C2-C8 alkynyl, cycloalkyl and aryl;
or R₁₁ and R₁₂ or R₁₂ and R₁₃ form together with the carbon atoms they are attached to C1-C5 alkyl substituted or unsubstituted, saturated or unsaturated 5 and 6 membered rings containing C, O and/or N atoms.

2. The use according to claim 1, wherein
R₁-R₂ is independently selected from the group consisting of H, Me, Et, vinyl, ethynyl, allyl, iso-Pr, n-Pr, propenyl, propynyl, 2-methylprop-1-enyl, methylcarboxylate, ethylcarboxylate, and acetyl;
R₃-R₅ is independently selected from the group consisting of H, Me, Et, iso-Pr, n-Pr, iso-Bu, n-Bu, tert-Bu, and sec-Bu; and
R₆-R₇ is independently selected from the group consisting of H, Me, Et; iso-Pr, n-Pr, iso-Bu, n-Bu, tert-Bu, sec-Bu, propenyl, propynyl, methoxymethyl, cyclopropyl, cyclopropylmethyl, cyclobutyl, methylcarboxylate, ethylcarboxylate, carbaldehyde, hydroxymethyl, 1,3-butadienyl, 2-methylallyl, vinyl, ethynyl, and acetyl;
or
R₁ and R₆, R₁ and R₃, R₁ and R₅, R₃ and R₅, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, while the other substituents have the same meaning as previously defined;
and the sum of all carbon atoms of R₁-R₇ is limited to 4;
or R₇ is —CR′₃R′₄—CR′₁R′₂—X′, wherein R′₁, R′₂, R′₃, R′₄, and X′ are selected from the corresponding groups of R₁, R₂, R₃, R₄, and X, and the sum of all carbon atoms of R₁-R₆ and R′₁-R′₄ is limited to 4;
and wherein
R₁₁ is selected from the group consisting of H, C1-C5 alkyl, OH, OMe, OEt, O-n-Pr, O-iso-Pr, hexanoyl, heptanoyl, octanoyl, propanoyl, 3-phenylpropanoyl, 5-methylhexanoyl, Cl, Br, phenyl, CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, para-tolyl;
R₁₂ is selected from the group consisting of H, OH, C1-C5 alkyl, C2-C5 alkenyl, and C1-C5 alkoxy;
R₁₃ is selected from the group consisting of H, branched or linear C1-C5 alkyl, branched or linear C2-C5 alkenyl, CHO, CO₂H, CO₂Me, CO₂Et, C(O)Me, C(O)Et, (CH₂)₂C(O)CH₃; (4-methyl-3,6-dihydro-2H-pyran-2-yl), CH₂—O-Me, CH₂—O-Et, CH₂—O-iPr, CH₂—OH, Cl, propanoyl, and CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, and para-tolyl;
R₁₄ is selected from the group consisting of H, OH, C1-C5 alkyl, C2-C5 alkenyl, and C1-C5 alkoxy;
R₁₅ is selected from the group consisting of H, C1-C5 alkyl, and Br;
or R₁₁ and R₁₂ or R₁₂ and R₁₃ form together with the carbon atoms they are attached to C1-C5 alkyl substituted or unsubstituted, saturated or unsaturated 5 and 6 membered rings containing C, O and/or N atoms.

3. A compound of formula (I) 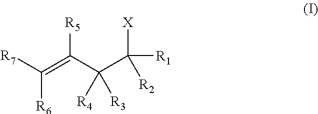
wherein
R₁-R₇ is independently selected from the group consisting of H₁, linear C1-C4 hydrocarbon groups and branched C1-C4 hydrocarbon groups, wherein the linear and branched C1-C4 hydrocarbon groups independently bear up to 2 heteroatoms selected from the group consisting of O, S and N, and bear up to one double bond or triple bond, and bearing up to one ring; or
R₁ and R₆, R₁ and R₃, R₁ and R₅, R₃ and R₅, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, while the other substituents have the same meaning as previously defined;
and the sum of all carbon atoms of R₁-R₇ is limited to 4;
or R₇ is —CR′₃R′₄—CR′₁R′₂—X′, wherein R′₁, R′₂, R′₃, R′₄, and X′ are selected from the corresponding groups of R₁, R₂, R₃, R₄, and X, while the other substituents have the same meaning as previously defined, and the sum of all carbon atoms of R₁-R₆ and R′₁—R′₄ is limited to 4;
and wherein X is representing the following fragment 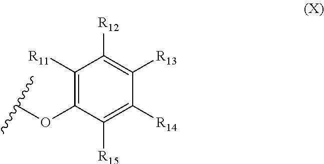
wherein
R₁₁-R₁₅ is independently selected from the group consisting of H, linear or branched C1-C5 alkyl, linear or branched C2-C5 alkenyl, linear or branched C2-C5 alkynyl, OH, C1-C4-alkoxy, CHO, C(O)Me, C(O)Et, hexanoyl, heptanoyl, octanoyl, propanoyl, 3-phenylpropanoyl, 5-methylhexanoyl, (CH₂)₂C(O)CH₃, (4-methyl-3,6-dihydro-2H-pyran-2-yl), CH₂—O-Me, CH₂—O-Et, CH₂—O-iPr, CH₂—OH, Cl, Br, phenyl, and CH(OR₁₆)(OR₁₇) wherein R₁₆ and R₁₇ are independently selected from the group consisting of C1-C5 alkyl, R₁₆ and R₁₇ form together with the carbon atoms of the chain they are attached to a 5 or 6 membered ring, C═N—OR₁₃ (R₁₃═C1-C5 alkyl), and CO₂R₁₉, wherein R₁₉ is selected from the group consisting of linear or branched C1-C8 alkyl, linear or branched C2-C8 alkenyl, linear or branched C2-C8 alkynyl, cycloalkyl and aryl;
or R₁₁ and R₁₂ or R₁₂ and R₁₃ independently form together with the carbon atoms they are attached to C1-C5 alkyl substituted or unsubstituted, saturated or unsaturated 5 and 6 membered rings containing C, O and/or N atoms;
with the proviso that the compound is not (Z)-1-(tert-butyl)-4-(hex-3-en-1-yloxy)benzene, (Z)-1-(hex-3-en-1-yloxy)-4-methylbenzene, (Z)-1-(hex-3-en-1-yloxy)-3-methylbenzene, (Z)-1-(hex-3-en-1-yloxy)-2-methylbenzene, or (Z)-(hex-3-en-1-yloxy)benzene.

4. The compound according to claim 3, wherein
R₁-R₂ is independently selected from the group consisting of H, Me, Et, vinyl, ethynyl, allyl, iso-Pr, n-Pr, propenyl, propynyl, 2-methylprop-1-enyl, methylcarboxylate, ethylcarboxylate, and acetyl;
R₃-R₅ is independently selected from the group consisting of H, Me, Et, iso-Pr, n-Pr, iso-Bu, n-Bu, tert-Bu, and sec-Bu; and
R₆-R₇ is independently selected from the group consisting of H, Me, Et; iso-Pr, n-Pr, iso-Bu, n-Bu, tert-Bu, sec-Bu, propenyl, propynyl, methoxymethyl, cyclopropyl, cyclopropylmethyl, cyclobutyl, methylcarboxylate, ethylcarboxylate, carbaldehyde, hydroxymethyl, 1,3-butadienyl, 2-methylallyl, vinyl, ethynyl, and acetyl;
or
R₁ and R₆, R₁ and R₃, R₁ and R₅, R₃ and R₅, R₅ and R₇, R₆ and R₇, R₁ and R₂, or R₃ and R₄, form together with the carbon atoms of the chain they are attached to a 5 or 6 membered ring bearing up to 2 heteroatoms selected from the group consisting of O, S and N, while the other substituents have the same meaning as previously defined;
and the sum of all carbon atoms of R₁-R₇ is limited to 4;
or R₇ is —CR′₃R′₄—CR′₁R′₂—X′, wherein R′₁, R′₂, R′₃, R′₄, and X′ can be are selected from the corresponding groups of R₁, R₂, R₃, R₄, and X, and the sum of all carbon atoms of R₁-R₆ and R′₁-R′₄ is limited to 4;
and wherein
R₁₁ is selected from the group consisting of H, C1-C5 alkyl, OH, OMe, OEt, O-n-Pr, 0-iso-Pr, hexanoyl, heptanoyl, octanoyl, propanoyl, 3-phenylpropanoyl, 5-methylhexanoyl, Cl, Br, phenyl, and CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, and para-tolyl;
R₁₂ is selected from the group consisting of H, OH, C1-C5 alkyl, C2-C5 alkenyl, and C1-C5 alkoxy;
R₁₃ is selected from the group consisting of H, branched or linear C1-C5 alkyl, branched or linear C2-C5 alkenyl, CHO, CO₂H, CO₂Me, CO₂Et, C(O)Me, C(O)Et, (CH₂)₂C(O)CH₃; (4-methyl-3,6-dihydro-2H-pyran-2-yl), CH₂—O-Me, CH₂—O-Et, CH₂—O-iPr, CH₂—OH, Cl, propanoyl, and CO₂R₁₉, wherein R₁₉ is selected from the group consisting of H, Me, Et, 2-methylpropyl, hexan-2-yl, 2-isopropoxyethyl, 3-methylbut-2-en-1-yl, n-pentyl, hexyl, benzyl, cyclohexyl, cis-3-hexenyl, 2-ethylhexyl, 3-methylhex-2-en-1-yl, 2-hept-4-enyl, 1-(3-methyl-2-hexenyl), (CH₂)₂-phenyl, and para-tolyl;
R₁₄=is selected from the group consisting of H, OH, C1-C5 alkyl, C2-C5 alkenyl, and C1-C5 alkoxy;
R₁₅ is selected from the group consisting of H, C1-C5 alkyl, and Br;
or R₁₁ and R₁₂ or R₁₂ and R₁₃ form together with the carbon atoms they are attached to C1-C5 alkyl substituted or unsubstituted, saturated or unsaturated 5 and 6 membered rings containing C, O and/or N atoms.

5. The compound of formula (I) according to claim 3, wherein the residue attached to the fragment X is selected from the group consisting of cis-3 hexenyl, trans-3-hexenyl, cis or trans-pentenyl, prenyl (3-methyl-3-butyl) and cyclopent-3-enyl.

6. A fragrance composition comprising at least one compound of formula (I) according to claim 3.

7. A consumer product comprising at least one compound of formula (I) according to claim 3 and a consumer product base.

8. A method to release a phenolic fragrant compound of formula (II), the method comprising exposing a compound of formula (I) as defined in claim 1 to ambient air in the presence or absence of light.

9. A method of making a compound of formula (I) according to claim 1, comprising the steps of:
a) providing a phenolic fragrant compound HX, which is a compound of formula (II) 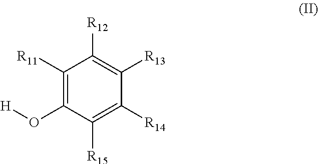
wherein
R₁₁-R₁₅ is independently selected from the group consisting of H, linear or branched C1-C5 alkyl, linear or branched C2-C5 alkenyl, linear or branched C2-C5 alkynyl, OH, C1-C4-alkoxy, CHO, C(O)Me, C(O)Et, hexanoyl, heptanoyl, octanoyl, propanoyl, 3-phenylpropanoyl, 5-methylhexanoyl, (CH₂)₂C(O)CH₃, (4-methyl-3,6-dihydro-2H-pyran-2-yl), CH₂—O-Me, CH₂—O-Et, CH₂—O-iPr, CH₂—OH, Cl, Br, phenyl, and CH(OR₁₆)(OR₁₇) wherein R₁₆ and R₁₇ are independently selected from the group consisting of C1-C5 alkyl, R₁₆ and R₁₇ form together with the carbon atoms of the chain they are attached to a 5 or 6 membered ring, C═N—OR₁₈ (R₁₈═C1-C5 alkyl), and CO₂R₁₉, wherein R₁₉ is selected from the group consisting of linear or branched C1-C8 alkyl, linear or branched C2-C8 alkenyl, linear or branched C2-C8 alkynyl, cycloalkyl and aryl:
or R₁₁ and R₁₂ or R₁₂ and R₁₃ may form together with the carbon atoms they are attached to C1-C5 alkyl substituted or unsubstituted, saturated or unsaturated 5 and 6 membered rings containing C, O and/or N atoms,
b) reacting it with an unsaturated alkylhalide or an unsaturated alcohol.

10. A method to confer, enhance, improve or modify the hedonic properties of a fragrance composition or a consumer product, the method comprising adding to said fragrance composition or consumer product at least one compound of formula (I) as defined in claim 1.
